SEQUENCE LISTING (16 sequences)

SEQ ID NO: 1 - DNA, 23 bp, synthetic construct
  source 1..23: mol_type = other DNA
tgtcccattcctctcatcaaaac

SEQ ID NO: 2 - DNA, 23 bp, synthetic construct
  source 1..23: mol_type = other DNA
tttacctcttcctgaaacttcct

SEQ ID NO: 3 - DNA, 23 bp, synthetic construct
  source 1..23: mol_type = other DNA
cccattcctctcatcaaaactca

SEQ ID NO: 4 - DNA, 23 bp, synthetic construct
  source 1..23: mol_type = other DNA
tcctttacctcttcctgaaactt

SEQ ID NO: 5 - DNA, 20 bp, synthetic construct
  source 1..20: mol_type = other DNA
tcccagaatgtcccattcct

SEQ ID NO: 6 - DNA, 23 bp, synthetic construct
  source 1..23: mol_type = other DNA
caaggtctagataaggtggttcc

SEQ ID NO: 7 - DNA, 23 bp, synthetic construct
  source 1..23: mol_type = other DNA
gcaaactatccatgaaactgtgc

SEQ ID NO: 8 - DNA, 20 bp, synthetic construct
  source 1..20: mol_type = other DNA
tttgccaagacctccagtgt

SEQ ID NO: 9 - DNA, 56 bp, synthetic construct
  source 1..56: mol_type = other DNA
acactctttccctacacgacgctcttccgatctgcaaactatccatgaaactgtgc

SEQ ID NO: 10 - DNA, 52 bp, synthetic construct
  source 1..52: mol_type = other DNA
gactggagttcagacgtgtgctcttccgatcttttgccaagacctccagtgt

SEQ ID NO: 11 - RNA, 20 nt, synthetic construct
  source 1..20: mol_type = other RNA
gactaatactattaccatcc

SEQ ID NO: 12 - RNA, 20 nt, synthetic construct
  source 1..20: mol_type = other RNA
gcttgataggttgtatatgt

SEQ ID NO: 13 - RNA, 20 nt, synthetic construct
  source 1..20: mol_type = other RNA
gtctgagctacagttaatat

SEQ ID NO: 14 - RNA, 20 nt, synthetic construct
  source 1..20: mol_type = other RNA
atttgtggcactgatcttag

SEQ ID NO: 15 - DNA, 337 bp, synthetic construct
  source 1..337: mol_type = other DNA
gtttcgctattcaaattggcttgataggttgtatatgttggtggggacaatggctgccatcatccatggagctgcactccctctcatgatgctggtttttggaaacatgacagctttgcaaatgcaggaatttcaagaaacaaaacttttccagttataattaatgaaagtaagtattatttgtggcactgatcttagaggtttgaagaaaaatctgttacttagaaggatgtttagccctcattaaatacattagtgtgtttatttctgggggaaaagggaggagtctgagctacagttaatattggtaataagtttgagaagataataatcataa

SEQ ID NO: 16 - DNA, 337 bp, synthetic construct
  source 1..337: mol_type = other DNA
caaagcgataagtttaaccgaactatccaacatatacaaccacccctgttaccgacggtagtaggtacctcgacgtgagggagagtactacgaccaaaaacctttgtactgtcgaaacgtttacgtccttaaagttctttgttttgaaaaggtcaatattaattactttcattcataataaacaccgtgactagaatctccaaacttctttttagacaatgaatcttcctacaaatcgggagtaatttatgtaatcacacaaataaagacccccttttccctcctcagactcgatgtcaattataaccattattcaaactcttctattattagtatt

(19) United States
(12) Patent Application Publication
(10) Pub. No.: US 2024/0060050 A1
(43) Pub. Date: Feb. 22, 2024

(54) CANINE HEPATIC ORGANOIDS

(71) Applicant: Iowa State University Research Foundation, Inc., Ames, IA (US)

(72) Inventors: Jonathan P. Mochel, Morlaas (FR); Karin Allenspach Jorn, Statham, GA (US); Vojtech Gabriel, Portland, ME (US); Christopher Zdyrski, Winterville, GA (US)

(21) Appl. No.: 18/469,214

(22) Filed: Sep. 18, 2023

Related U.S. Application Data
(63) Continuation of application No. PCT/US22/20768, filed on Mar. 17, 2022.
(60) Provisional application No. 63/200,614, filed on Mar. 18, 2021.

(51) Int. Cl. C12N 5/071

(52) U.S. Cl. CPC C12N 5/0671; C12N 2501/119; C12N 2501/12; C12N 2501/155; C12N 2501/39; C12N 2501/42; C12N 2503/04; C12N 2506/14; C12N 2510/00; C12N 2513/00

(57) ABSTRACT

The present invention relates to hepatic organoids and uses thereof. The compositions include models for the study of developmental biology of the liver and other epithelial tissues, drug discovery and toxicity screening, infectious disease biology of various infectious agents, and personalized medicines. Methods for seeding canine intestinal organoids, maintaining an organoid monolayer, and monitoring monolayer integrity are provided. Methods and systems for culturing, freezing, and recovering the frozen organoid cells are also provided. The hepatic organoids may also be used to treat a subject in need, or for identifying a preferred therapeutic agent.

### CROSS REFERENCE TO RELATED APPLICATIONS

This is a continuation application of International Patent Application No. PCT/US2022/20786, filed on Mar. 17, 2022, which claims the benefit of priority of U.S. Provisional Patent Application No. 63/200,614, filed Mar. 18, 2021, each of which is incorporated herein by reference in their entirety.

### STATEMENT REGARDING FEDERALLY SPONSORED RESEARCH

This invention was made with Government support under Contract Number IOS2127995 and Contract Number IIP1912948 awarded by the National Science Foundation, and under Contract Number W81XWH-20-1-0620 awarded by the Department of Defense. The government has certain rights in the invention.

### REFERENCE TO SEQUENCE LISTING SUBMITTED ELECTRONICALLY

The instant application contains a Sequence Listing which has been submitted electronically in XML format and is herein incorporated by reference in its entirety. Said XML copy, created on Sep. 14, 2023, is named “P13486US01_SequenceListing.xml” and is 15,911 bytes in size.

### FIELD OF THE INVENTION

Described herein are compositions for the growth of canine epithelial organoids and methods of using such organoids. Transwell systems and methods for culturing, freezing, and recovering organoids are also provided.

### BACKGROUND OF THE INVENTION

Animal models, particularly murine models, have been employed extensive to study diseases due to cost effectiveness, ethical considerations, and the easy accessibility to genetically engineered technology. Despite the wide use of mouse models in biomedical research, the translational value of mouse studies for human disease remains controversial. In addition, mice and other rodents often fail to adequately represent the human condition, as well as drug response in toxicity and efficacy studies. Given the high failure rate of drugs from discovery and development through the clinical trial phase (i.e., more than 90%), there is now a critical need for better animal models for preclinical studies.

Models for larger animals, such as the dogs, are typically more representative than mice as they have a relatively large body size, longer life span, more closely resemble human GI physiology, and develop spontaneous, analogous diseases including inflammatory bowel disease (IBD) and colorectal cancer (CRC). Dogs have been used as an animal model for human health and disease from the ancient to the modern era. The dog is still considered to be superior to non-rodent mammalian animal models for pharmaceutical research and is preferred by the FDA for initial safety data of drugs for human use. Although the dog has contributed immensely to the advancement of medical knowledge in the past, the use of the dog in medical research has declined in recent years due to the emotional perceptions among the public and ensuing ethical concerns with canine research.

Currently, there are a limited number of canine-specific primary cell lines to investigate epithelial physiology, such as bile duct physiology, ex vivo or in vitro. For example, for hepatic physiology the well-characterized immortalized cell lines including the Madin-Darby canine kidney (MDCK) cells do not accurately model hepatic epithelial interactions in the dog due to their origin from immature kidney cells. Recently, isolated primary canine hepatic epithelial cells have been immortalized with a temperature-sensitive mutant of the Simian Virus 40 large tumor antigen (SV40 T-Ag). Although this cell line can be grown on a monolayer, the SV40 T-Ag may initiate pathways which could provide spurious, non-physiologic findings ex vivo given its tumorigenic cell line origin.

Canine GI organoids arose as a model to bridge the gap in the drug development pipeline by providing a more representative in vitro model to test drug efficacy and toxicity in preclinical studies, as well as an innovative screening tool in drug discovery, while also reducing the number of animals needed for in vivo studies. Thus, the ultimate goal of the herein disclosed research is to culture canine hepatic organoids from healthy and diseased dogs to develop better therapeutic strategies and personalized medicine for both animal and human health.

Stem cell-derived 3D organoids have emerged as a cutting-edge cell culture technology to study the developmental biology of the intestines, brain, stomach, and liver; drug discovery and toxicity screening; drug testing for personalized medicine; infectious disease biology of the microbiome, including bacteria and viruses; and regenerative medicine. Organoids are collections of organ-specific cell aggregates derived from either primary tissue or stem cells that are capable of organ-like functionality in an in vitro environment. The 3D organoid model better reproduces the in vivo biology, structure, and function, as well as genetic and epigenetic signatures of original tissues, unlike widely used two-dimensional (2D) cell monolayer models that utilize cancer and immortalized cell lines.

Organoids may be developed from either embryonic or induced pluripotent derived stem cells (iPSC) or organ-specific adult stem cells (ASC). Organoids derived from ASCs are generated without genetic transduction by transcription factors, unlike organoids derived from iPSCs, thus providing a more physiologically relevant in vitro model than iPSC-derived organoids. ASC-derived organoids are a functional model that can be differentiated to replicate the in vivo adult environment and can be safely transplanted into animals and humans.

### SUMMARY OF THE INVENTION

Compositions for the growth of canine epithelial organoids and methods of using such organoids are described herein. Said compositions include models for the study of developmental biology of the liver and other epithelial tissues, drug discovery and toxicity screening, drug testing for personalized medicine, infectious disease biology of viruses, bacteria and other infectious agents, the interaction of the microbiome with the epithelial cell layer, cancer, regenerative medicine, and personalized medicine. Methods and systems for culturing, freezing, and recovering of the frozen cells are also provided.

An advantage of the invention is to provide models which more closely reflect the physiological state of a subject or subjects than the currently available model systems. It is an advantage of the present invention that the models may be further genetically modified, for example to alter the expression of P-glycoprotein. It is also an advantage of the models that they either represent a single time point or by taking advantage of the shorter lifespan of canines compared humans to be create longitudinal canine models for chronic human diseases. It is a further advantage of the models that both healthy and diseased models may be made from the same animal.

In an embodiment, the present invention provides stem cell derived hepatic organoid models. In some embodiments the organoids are hepatocyte organoids. In other embodiments, the organoids are cholangiocyte organoids. The stem cells are grown in media that first promotes stem cell expansion and then a media that allows their differentiation into their complex 3D structures formed by cholangiocytes and hepatocytes. In an embodiment, the organoids are spherical and grown in solution. In a further embodiment, the cells are grown in an extracellular matrix. In another embodiment, the organoids are grown flat on a membrane or plate to provide ready access to the lumen of the organoid. In other embodiments, the hepatic stem cell derived model is a two-dimensional monolayer of an organoid grown on a permeable membrane, such as, but not limited to, a TRANSWELL® membrane. In another embodiment are methods for growing the organoids in either spherical form. In still yet another embodiment are methods for growing the organoid on a substrate.

In an embodiment, the present invention provides adult stem cell derived organoid models for physiological and disease research. In a further embodiment, a healthy control is compared to a diseased sample. In a further embodiment, the healthy control originates from the same animal as the diseased sample. In another embodiment, the healthy sample is derived from a different animal than the diseased sample. In an embodiment, the disease is cancer or inflammatory bowel disease.

In an embodiment, the present invention provides adult stem cell derived organoid models for testing drug absorption, efficacy, and safety. In a further embodiment, the model uses P-glycoprotein (P-gp) transport to study drug absorption. In some embodiments, the stem cells are derived from control or healthy subjects. In other embodiments, the stem cells are derived from subjects with a disease, or which have been genetically modified to alter the expression of P-glycoprotein. In some embodiments, the genetic modification comprises a knock-in mutation of an ABCB1-1Δ mutation. Models made from control or healthy subjects may be used to test and screen drugs for normal physiological absorption while organoids derived from diseased or genetically modified subjects may be used to test and screen drugs under various physiological conditions.

In an embodiment, the organoid models and methods of use described herein provide three-dimensional culture conditions, including passaging, freezing, and recovery of the frozen organoids. These models may be used for screening of potential therapeutic drugs and screening of drug responses in ex vivo models. The embodiments provide a canine-specific system for testing P-gp affinity in in therapeutic drug development. As referred to herein, drug screening and development can include pharmacotherapeutic effects, bioavailability, elimination, efficacy, and various safety effects, among others. In an embodiment, the organoids are able to predict clinical responses, such as efficacy and/or adverse effects, and thereby enable designing therapies, including therapies for healthy subjects, diseased subjects, and/or any subject requiring personalized treatment. These embodiments include the optimization of individualized medicine and testing of the bioavailability of drugs across the hepatocytes or cholangiocytes, bile duct epithelial cells.

In an embodiment the methods include administering to a model a drug and a P-gp interacting compound; measuring the rate of transport of the drug across P-gp; and comparing the rate of transport to a model lacking the P-gp interacting compound. If said drug is a substrate for P-gp, then the P-gp interacting compound is preferably an inhibitor to control for the effect on transport of P-gp. If the drug is an inhibitor or inducer of P-gp, then the P-gp interacting compound is preferably a P-gp substrate in order to measure the effects of the drug on the transport function of P-gp. In a further embodiments, additional inhibitors, inducers, or substrates may be administers.

In another embodiment the present invention includes systems using the models to test or screen a drug for P-glycoprotein transport comprising the model of the invention, a P-gp interacting compound; and a way of detecting the transportation. In some embodiments the way of detecting the transportation is a change in fluorescence. In other embodiments the way of detecting the transportation may be a binding assay, such as an antibody detection system. In other embodiments the way of detecting the transportation may be through high performance liquid chromatography (HPLC) and mass spectrometry (MS). In still other embodiments, detection may be through staining. While multiple embodiments are disclosed, still other embodiments will become apparent to those skilled in the art from the following detailed description, which shows and describes illustrative embodiments. Accordingly, the drawings and detailed description are to be regarded as illustrative in nature and not restrictive.

The disclosure further provides standard operating procedures for the culture of canine hepatic organoids on Transwell inserts. A first transwell seeding protocol (TSP) describes the experimental methods for dissociating and seeding canine organoids on inserts. Canine organoid isolation, culture, and harvest are also described. Methods for general upkeep of the canine hepatic organoid 2D monolayer on a Transwell are also disclosed in a monolayer maintenance protocol. Additionally, the disclosure includes methods to assess the structural integrity of a monolayer via transepithelial electrical resistance (TEER) measurements and light microscopy. Finally, a permeability experimental protocol describes the tasks directly preceding an experiment, including in vitro validation of experimental results. A variety of media for use in the above protocols is also described.

It is therefore an object of this disclosure to provide hepatic stem cell derived models for studying canine hepatic tissue. It is also an object of the disclosure to provide methods of making, freezing, and recovering of hepatic organoids. It is also a further objective to provide methods for using genetically modified organoids for regenerative or personalized medicine.

It is a further object of the disclosure to provide methods of using the models for testing drugs and performing hepatic research.

It is another object of the disclosure to provide systems using the models for drug testing and screening and for the studying of hepatic physiology, both in healthy and diseased states, and in different environmental or dietary regimes. These studies may lead to the use of the canine hepatic organoids for personalized medicine.

Overall, the canine organoid model, combined with the Transwell technology described herein, overcomes limitations associated with 2D experimental models, thereby improving upon the reliability of predictions pertaining to the apparent oral permeability of therapeutic drug candidates both in the canine and human patients.

Other objects, aspects and advantages of this invention will be apparent to one skilled in the art in view of the following disclosure, the drawings, and the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows LGR5 positive hepatic organoid cells on day 2 of differentiation.

FIG. 1B shows LGR5 positive hepatic organoid cells on day 3 of differentiation. FIG. 1C shows LGR5 positive hepatic organoid cells on day 6 of differentiation. FIG. 1D shows LGR5 positive hepatic organoid cells on day 7 of differentiation.

FIG. 2A shows KRT7 positive hepatic organoid cells on day 2 of differentiation.

FIG. 2B shows KRT7 positive hepatic organoid cells on day 7 of differentiation. FIG. 2C shows KRT7 positive hepatic organoid cells on day 11 of differentiation.

FIG. 3A shows CYP3A12 positive hepatic organoid cells on day 7 of differentiation. FIG. 3B shows CYP3A12 positive hepatic organoid cells on day 11 of differentiation.

FIG. 4A shows CYP3A4 positive hepatic organoid cells on day 7 of differentiation. FIG. 4B shows CYP3A4 positive hepatic organoid cells on day 7 of differentiation.

FIG. 5 shows the workflow of the transwell seeding protocol (“TSP”). TSP includes insert pre-coating, incubation, canine organoid dissociation, and canine organoid seeding steps.

FIG. 6 shows the composition of twenty tested hepatocyte differentiation media and four control media. Components were either added (HGF, FGF10, ROCKi/CHIR99021 (GSK3 inhibitor)=R/G, BMP7, DAPT, FGF19, Dex) or removed (R/G, R-spondin, Nico). Day of addition or removal is indicated by cross hatching. Additional four control media were included in the experiment.

FIG. 7 shows a summary of the most important media components for hepatocyte differentiation displaying the pathways and the overall outcome. Stimulation (green arrow), inhibition (red arrow), hypothesized interaction (dashed arrow), and unknown pathways (question mark) are denoted. FGF19—Fibroblast Growth Factor 19; HGF—Hepatocyte Growth Factor; FGF10—Fibroblast Growth Factor 10; BMP7—Bone Morphogenetic Protein 7; DAPT—N-[N-(3,5-Difluorophenacetyl)-L-alanyl]-S-phenylglycine t-butyl ester; PI3K-AKT—Phosphatidylinositol-3-Kinase and Protein Kinase B pathway; Ras/Raf/MAPK—Ras/Raf/mitogen-activated protein kinase cascade; STAT—Signal Transducer and Activator of Transcription pathway; TNF-α—Tumor Necrosis Factor-alpha; IL-6—Interleukin 6.

FIG. 8 shows images of treatment groups and control captured on days 1, 5, 7, 11, and 14 via light microscopy. The scale bars are in vim.

FIGS. 9A-9B show albumin experimental results. 9A shows a summary of highest yielding albumin production media from the initial experiment measured using ELISA on day 14 of differentiation. These media were selected for the final experiment. 9B shows a summary of the albumin measurement experiment representing 20 different media measured based on the individual components and their combination. Albumin yields less than 10Ong of albumin/1000 cells (white circle), and a yield of 400 ng of albumin/1000 cells (grey circle) is indicated. Samples, where R-spondin was removed on day 5 of the experiment are indicated with blue squares, while media outside the rectangle represent media supplemented with R-spondin for the whole experiment run.

FIGS. 10A-10F show histological staining of organoids grown in experimental and control media. 10A shows images of Hematoxylin & Eosin (H&E) staining for morphology characterization. 10B shows Images of Periodic Acid-Schiff (PAS) staining showing glycogen accumulation. 10C shows images of PicroSirius Red stain showing the presence of connective tissue. 10D shows images of Masson's Trichrome identifying collagen. 10E shows images of Periodic Schiff-Methenamine Silver (PASM) stain for basement membrane. 10F shows images of the Prussian Blue stain. Images were captured at 60× objective magnification. The scale bars are in vim.

FIG. 11A shows the histological ratio of cholangiocytes vs. mature hepatocytes in tested media. FIG. 11B shows comparison of M01 and control supernatant based on proteins with highest and lowest abundance ratios acquired using label-free relative quantitative proteomics technique. FIG. 11C shows P-glycoprotein detected via IF captured at 20× resolution (top row) and 60× (bottom row) FIG. 11D shows representative images captured via transmission electron microscopy.

FIG. 12 shows the workflow of dissociation, transfection, and screening of genetic mutants. Organoids were first grown, dissociated, and then electroporated. After electroporation, a control cuvette with a GFP plasmid was screened to confirm successful electroporation. Once electroporated organoids recovered and grew, a subset of them were taken for DNA extractions and DNA sequencing.

FIG. 13A shows an agarose gel showing both extracted DNA and PCR amplification using the MDR1 P5 primer pair showing the simultaneous cutting of both gRNAs resulting in a loss of ˜271 bp in the truncated sequence. FIG. 13B shows the editing efficiency of the five samples. No incorporation of the HDR strand was seen in the Passage Control sample and minimal incorporation using the Anti Strand. Successful incorporation was seen more than twice as common in Sense Cold than Sense Hot.

FIG. 14 shows the editing method using two gRNAs and one ssDNA HDR template. All components were pooled in an electroporation cuvette with a template missing four base pairs to accurately mimic the ABCB1-1Δ mutation found in canines. The ssDNA repair template can be changed to rescue the mutation without affecting the gRNAs cut sites.

Various embodiments of the present invention will be described in detail with reference to the drawings, wherein like reference numerals represent like parts throughout the several views. Reference to various embodiments does not limit the scope of the invention. Figures represented herein are not limitations to the various embodiments according to the invention and are presented for exemplary illustration of the invention.

### DETAILED DESCRIPTION

The present invention relates to methods and compositions for the growth of hepatic organoids for the study of drugs, including oral drug P-glycoprotein (P-gp) mediated absorption in dogs. The embodiments are not limited to particular models, methods of making the models, using the models for drug testing or screening, and compositions, which can vary and are understood by skilled artisans.

It is further to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form. Numeric ranges recited within the specification are inclusive of the numbers within the defined range. Throughout this disclosure, various aspects are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range (e.g., 1 to 5 includes 1, 1.5, 2, 2.75, 3, 3.80, 4, and 5).

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments without undue experimentation, but the preferred materials and methods are described herein. In describing and claiming the embodiments, the following terminology will be used in accordance with the definitions set out below.

The term “about,” as used herein, refers to variation in the numerical quantity that can occur, for example, through typical measuring and liquid handling procedures used for making concentrates or use solutions in the real world; through inadvertent error in these procedures; through differences in the manufacture, source, or purity of the ingredients used to make the compositions or carry out the methods; and the like. Whether or not modified by the term “about”, the claims include equivalents to the quantities.

The term “actives” or “percent actives” or “percent by weight actives” or “actives concentration” are used interchangeably herein and refers to the concentration of those ingredients involved in cleaning expressed as a percentage minus inert ingredients such as water or salts.

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

As used herein “organoids” refer to ex vivo models that are grown from adult stem cells to provide structures that resemble an organ in culture.

As used herein, the term “basal media” refers to a culture media that lacks some supplements that may be required for cell growth.

As used herein, the term “complete media” refers to a culture media that contains all the supplements to supports cell growth.

As used herein, the term “differentiation media” means any media that induces a stem cell, for example an induced pluripotent stem cell or an adult derived stem cell, to differentiate into the desired epithelial cells comprising the organoids.

As used herein the term “protecting media” refers to a differentiation media which inhibits cell death during cell culture.

As used herein, the term “freezing media” means any media in which the organoids may be frozen in and then recovered.

As used herein, the term “P-glycoprotein interacting compound” or “P-gp interacting compound” is any compound that functions as an inhibitor, inducer, or substrate for P-gp. An inhibitor may reduce the transport ability of P-gp, an inducer may increase the transport of P-gp, and a substrate may be transported by P-gp.

As used herein “antibodies” and like terms refer to immunoglobulin molecules and immunologically active portions of immunoglobulin (Ig) molecules, i.e., molecules that contain an antigen binding site that specifically binds (immunologically reacts with) an antigen. These include, but are not limited to, polyclonal, monoclonal, chimeric, single chain, Fc, Fab, Fab′, and Fab₂ fragments, and a Fab expression library. Antibody molecules relate to any of the classes IgG, IgM, IgA, IgE, IgD, which differ from one another by the nature of heavy chain present in the molecule. These include subclasses as well, such as IgG1, IgG2, and others. The light chain may be a kappa chain or a lambda chain. Reference herein to antibodies includes a reference to all classes, subclasses, and types. Also included are chimeric antibodies, for example, monoclonal antibodies or fragments thereof that are specific to more than one source, e.g., a mouse or human sequence.

The term “pharmaceutical agent” or “drug” refers to a chemical compound or composition capable of inducing a desired therapeutic or prophylactic effect when properly administered to a subject.

The term “sample” as referred to herein means an isolated part of an animal. Samples can include, but are not limited to, tissue sections, stem cells, cancerous cells, and tissue biopsies.

The term “subject” as used herein refer to a human or mammalian animal. The mammalian animal may include carnivores/omnivores or herbivores. Carnivores/omnivores may include canines, pigs, rodents, or felines.

The term “substantially free” as used herein refers to the amount of a compound may be present in a composition in so low as to not have a measurable effect. It should be noted that the compound may be present in the composition, for example, a specific growth factor is not added to a differentiation media may still be present in an organoid culture due to the organoid itself producing the growth factor.

The methods, compositions, and systems may comprise, consist essentially of, or consist of the components and ingredients as well as other ingredients described herein. As used herein, “consisting essentially of” means that the methods and compositions may include additional steps, components or ingredients, but only if the additional steps, components or ingredients do not materially alter the basic and novel characteristics of the claimed methods and compositions.

The methods, compositions, and systems may be substantially or essentially free of components and ingredients. As used herein, “substantially free” and “essentially free” mean that a component or ingredient may be present in the methods, compositions, or systems, but do not contribute any property to the methods, compositions, or systems.

3D Organoid Model and Transwell Protocols

The 3D Organoid model better reproduces the in vivo biology, structure, and function, as well as genetic and epigenetic signatures of original tissues, unlike widely used two-dimensional (2D) cell monolayer models that utilize cancer and immortalized cell lines.

Organoids may be developed from stem cells, such as, but not limited to, embryonic, induced pluripotent derived stem cells (iPSC), or organ-specific adult stem cells (ASC). Organoids derived from ASCs are generated without genetic transduction by transcription factors, unlike organoids derived from iPSCs, thus providing a more physiologically relevant in vitro model than iPSC-derived organoids. ASC-derived organoids are a functional model that can be differentiated to replicate the in vivo adult environment and can be safely transplanted into animals and humans. Hepatic stem cells may be differentiated into either hepatocytes or cholangiocytes, and organoids of each may be made following the methods disclosed herein.

Once the stem cells are isolated, they may then be grown in an extracellular matrix using a media appropriate to allow for the desired differentiation. The extracellular matrix may be a natural or synthetic extracellular matrix.

Examples of natural extracellular matrices include, but are not limited to, solubilized basement membrane preparations from Engelbreth-Hold-Swarm mouse sarcoma (MATRIGEL®), collagen, fibrin, or vitronectin.

Synthetic extracellular membranes are generally hydrogels composed of crossed linked polyethylene glycol (PEG) (for example see Nguyen et al., 2017, Versatile synthetic alternatives to MATRIGEL® for vascular toxicity screening and stem cell expansion, Nat Biomed Eng., 1: doi:10.1038/s41551-017-0096, herein incorporated by reference in its entirety). Hydrogel based extracellular matrices may provide benefits over naturally occurring extracellular matrices because the formation may be better controlled, leading to lowered lot to lot variability in desired properties.

In an embodiment, canine organoids are derived from adult hepatic stem cells. In some embodiments, the stem cells are derived from the liver. In some embodiments, the stem cells are derived from bile duct cells. In some embodiments, the organoids are derived from healthy tissues. In other embodiments, the organoids are derived from diseased tissues, such as but not limited to cancer.

The organoids may be produced from a human or an animal. More preferably, the organoids are produced from a carnivore, and even more preferably from a canine. In a more preferred embodiment, the organoids are derived canine epithelial cells.

In some embodiment, the organoids are produced from epithelial tissue making the liver or the lining of the bile duct. To produce the 3D cultures of canine hepatic organoids, leucine-rich repeat-containing G protein-coupled receptor 5 (Lgr5)-positive stem cells. Additionally, in some embodiments, the cells collected may be hepatic tumors.

The epithelial organoids of the present disclosure may be cultured from various sized samples of tissue. By way of nonlimiting example, for the organoids, large whole tissue sections or from much smaller endoscopic biopsy samples from a subject using a relatively non-invasive procedure. The large whole tissue sections may be from about 1 cm to about 20 cm, from about 2.5 cm to about 15 cm, or from about 5 cm to about 10 cm. The smaller samples may be 1 mm or less, 2 mm or less, or 3 mm or less in size. The collection of the hepatic tissue may be collected in any way known in the art. For example, the tissue may be collected from living or recently euthanized subjects.

For whole tissue sections, the tissue may then be immediately placed into a wash medium, such as, but not limited to, phosphate buffered saline (PBS) with about 1 mM to about 3 mM N-acetylcysteine, and vigorously shaken from about 3 to about 20 times, from about 5 to about 15 times, or from about 10 to about 15 times. The wash may be repeated about 3 times, about 4 times, or about 5 times or more to remove excess mucus and other debris. After washing, the cleaned tissues may be transferred to an appropriate culture media without growth factors. While any appropriate media may be used, in a preferred embodiment, the media is complete media without growth factors (abbreviated as CMGF−) as described in the Organoid Media section and incubated on ice.

Alternatively, a tissue sample may then be collected from hepatic tissue biopsy by any means known in the art. This may allow up to about 15 or more hepatic biopsies to be obtained from healthy or diseased canine subjects under general anesthesia. Collected biopsies may be placed in complete media, such as, but not limited to, CMGF− medium, on ice and subjected to mechanical cleansing as described above.

Both whole tissue samples and biopsies are typically cut into small pieces, from about 0.5 mm to about 5 mm, from about 1mm to about 3 mm, or from about 1 to about 2 mm in thickness with a scalpel and washed at least once, at least about 5 times, or at least about 10 times using a chelating solution. In a preferred embodiment, the chelating solution is a complete chelating solution (1× CCS) comprising from about 0.4 to about 0.6 g, from about 0.45 to about 0.55 g, or from about 0.48 to about 0.52 g Na₂HPO₄-2H₂O, from about 0.45 to about 0.65 g, from about 0.50 to about 0.6 g, or from about 0.5 to about 0.55 g KH₂PO₄, from about 2.3 to about 3.4 g, from about 2.5 to about 3.2 g, or from about 2.7 to about 3 g NaCl, from about 0.05 to about 0.75 g, from about 0.055 to about 0.07 g, or from about 0.58 to about 0.65 g KC1, from about 6.25 to about 9 g, from about 6.5 to about 8.5 g, or from about 7 to about 8 g Sucrose, and from about 4 to about 6 g, from about 4.5 to about 5.5 g, or from about 4.75 to about 5.25 g D-Sorbitol in about 500 mL water and supplemented from about 40 to about 60 μM, from about 45 to about 55 μM, or from about 50 to 55 μM DTT. While one skilled in the art will appreciate that salt solutions may be stored in concentrated form and then diluted, in a preferred embodiment, the 1× completely chelating solution may consist of a 1:5 diluted 5× CCS diluted in culture grade water, such as Milli-Q H2O water. To prevent adherence of the cells and allow for a higher yield of cells, plastic and glass ware may be pre-wetted with 1% bovine serum albumin (BSA) throughout the procedure.

Samples may then be incubated with 1× CCS containing from about 10 to about 50 mM, from about 15 to about 40 mM, or from about 20 to about 30 mM of a chelator, such as, but not limited to, methyl glycine diacetic acid (MGDA), glutamic acid N,N-diacetic acid (N.N-dicarboxymethyl glutamic acid tetrasodium salt, GLDA), nitrilotriacetic acid (NTA), diethylene triamine pentaacetic acid (DTPA), ethylenediaminetetraacetic acid (EDTA), Ethylenediamine-N,N′-disuccinic acid (EDDS), N-(1,2-dicarboxyethyl)-D,L-aspartic acid (IDS) and N-(2-hydroxyethyl)iminodiacetic acid (EDG), and salts thereof, for about 30 to about 90 minutes, for about 40 to about 80 minutes, or for about 45 to about 75 min at 4° C. on 20, 24 rpm mixer/rocker (Fisher). In a preferred embodiment, the chelator is EDTA.

After chelation, release of the cells may be augmented by trituration and/or mild vortexing in cell culture supernatant (CCS). Additional trituration and/or mild vortexing may be carried out after with the addition of fetal bovine serum (FBS; Atlanta Biologicals) to maximize release. Large fragments, such as tissue fragments, may then be allowed settled to the bottom of the tube, and the supernatant, containing the cells of interest, may then be transferred to a new conical tube and sufficiently centrifuged, for example at about 100 g, at about 125 g, at about 150 g, or about 175 g at 4° C. for about 3 minutes, for about 4 minutes, or for about 5 minutes. The pellet may then be washed with about 5 mL, about 7.5 mL, or about 10 mL complete medium, preferably CMGF−, and then sufficiently centrifuged, such as at about 60 g, at about 70 g, or about 80 g at 4° C. for about 3 minutes, for about 4 minutes, or for about 5 minutes. The pellet is then resuspended in 2 mL complete medium, and the approximate number of cells of interest isolated may be calculated using a hemocytometer.

In some embodiments, the organoids are then genetically modified using any known technique in the art. Examples of genetic modification include DNA modification, such as but not limited to non-homologous end joining (NHEJ), homologous repair (HR) with or without the mediation of a nuclease, such as, but not limited to, Cas variants, TALEN, meganucleases, or Zinc Fingers; or RNA modifications, such as, but not limited to, RNAi, LEAPER, or Cas mediated. PCR methods, such as site directed mutagenesis may also be used for the stem cells. Transient or stable transfection with an interfering RNA may also be used to alter RNA expression in the organoids. In some embodiments, the genetic modification may be used to increase or decrease the expression of a desired protein, such as P-gp for testing drug transfer or a transporter for testing uptake of different environmental factors, or the genetic modification may alter the function of a desired protein, for example, so that P-gp or a transporter becomes resistant or susceptible to its substrate, a novel substrate, or a drug, for example, by changing the pocket size or binding sites.

The hepatic cells may then be seeded into a well comprising an appropriate extracellular matrix. In a preferred embodiment, from about 20 to about 200, from about 30 to about 150, or from about 50 to about 100 cells may be seeded in each well of a 24-well plate, wherein each well comprising about 20 μL, about 30 μL, or about 40 μL of extracellular matrix and incubated at 37° C. for about 10 minutes. However, one skilled in the art will appreciate any sized culture system may be used and the number of cells and reagents may be scaled appropriately.

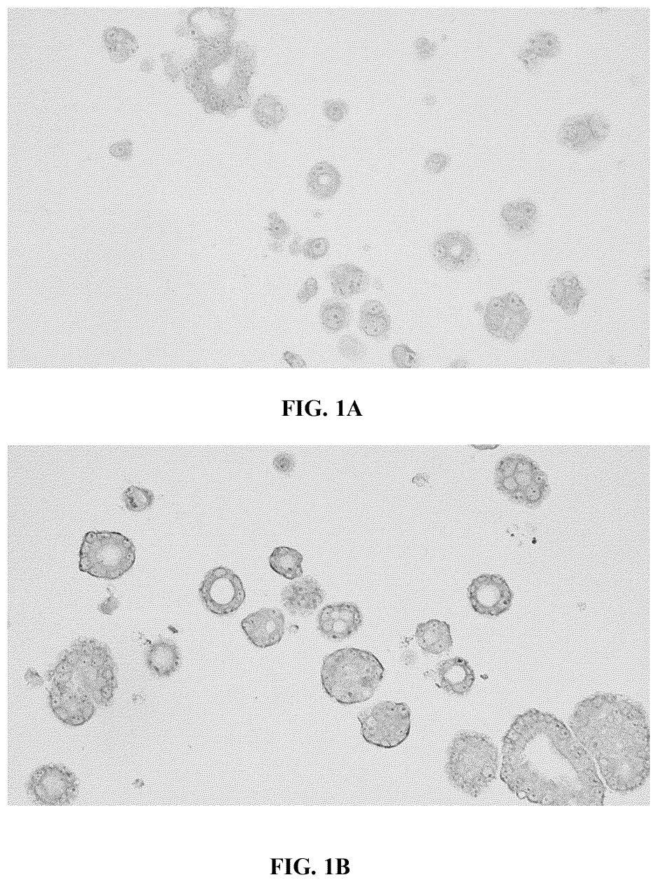

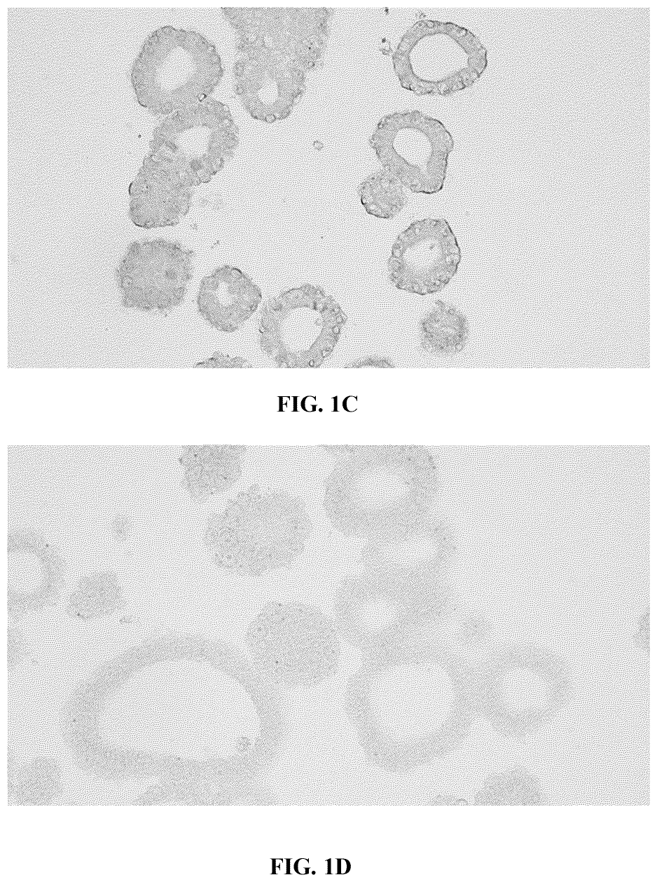

The hepatic cells may then be differentiated in the wells by adding a differentiation media. As shown in FIG. 1A, LGR5 positive hepatic organoid cells on day 2 are weakly differentiated or not differentiated. FIG. 1B shows LGR5 positive hepatic organoid cells on day 3 of differentiation, showing partial differentiation. FIG. 1C shows LGR5 positive hepatic organoid cells on day 6 of differentiation, showing moderate differentiation. FIG. 1D shows differentiated LGR5 positive hepatic organoid cells on day 7.

A preferred embodiment of a differentiation media comprises a complete medium with growth factors (abbreviated as CMGF+) as taught in the Organoid Media section. In a further embodiment, inhibitors may be added to the culture, forming a protective media as described in more detail in the Organoid Media section, and the organoids are incubated at 37° C. For carnivores, the protective medium with rho kinase (ROCK) and various glycogen synthase kinase 3 (GSK-3), such as GSK3β, inhibitors may be used from about 1 days to about 4 days of hepatic stem cell culture and may enhance stem cell survival and prevent apoptosis. In a preferred embodiment, CHIR99021, an inhibitor of GSK-3, in combination with Y-27632, an inhibitor of ROCK. The inhibitors may only be added temporarily to the media for the first 2 days after isolation of hepatic cells for organoids to culture and then removed. The short-term addition of the GSK-3 inhibitor, preferably CHIR99021, may enhance the initial survival and facilitated long-term propagation of organoid. Surprisingly, including Wnt3a in the media prevented colony forming efficiency and is not required for hepatic organoid survival, and the media is preferably substantially free of Wnt3a. Removal of the ROCK and GSK-3 inhibitors from the media after the first 2 days of culture may improve differentiation of the canine organoids.

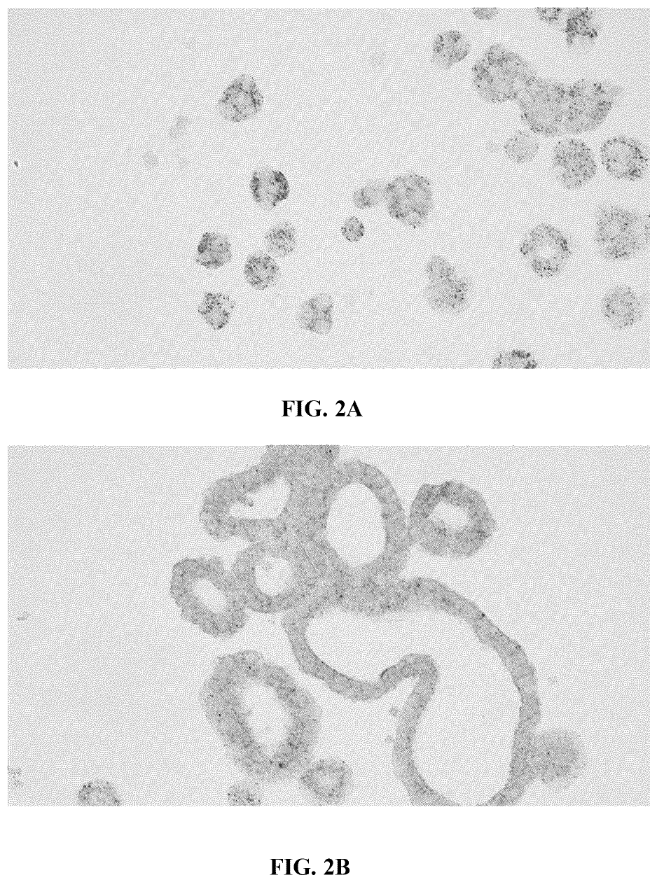

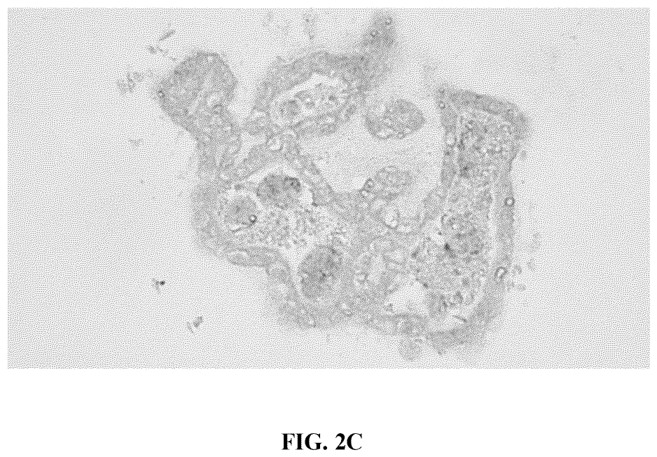

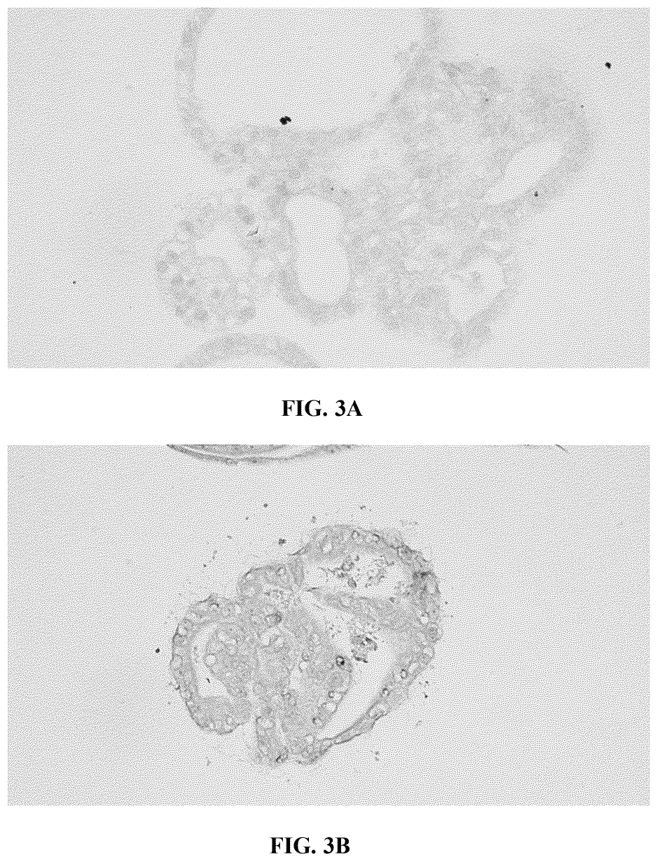

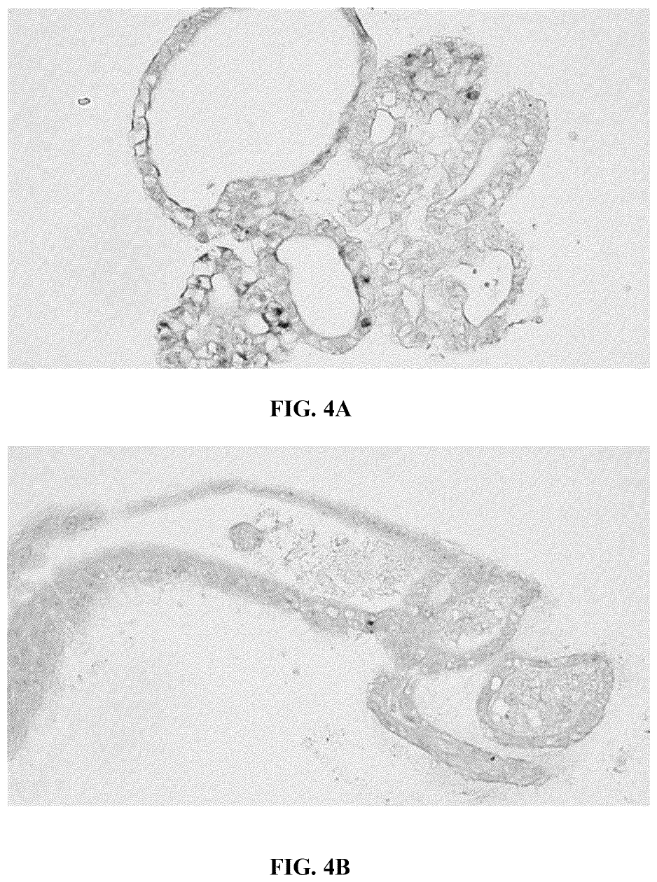

FIG. 2A shows KRT7 positive hepatic organoid cells on day 2 of differentiation. FIG. 2B shows KRT7 positive hepatic organoid cells on day 7 of differentiation. FIG. 2C shows KRT7 positive hepatic organoid cells on day 11 of differentiation. FIG. 3A shows CYP3A12 positive hepatic organoid cells on day 7 of differentiation. FIG. 3B shows CYP3A12 positive hepatic organoid cells on day 11 of differentiation. FIG. 4A shows CYP3A4 positive hepatic organoid cells on day 7 of differentiation. FIG. 4B shows CYP3A4 positive hepatic organoid cells on day 7 of differentiation.

The differentiation media, preferably CMGF+ medium, may be replenished as needed, for example every 2 days. One skilled in the art will appreciate that the changing of color of the basal media, if it contains phenol red, will signal the time to change the media. Culture may be maintained until the hepatic organoids are completely differentiated. To maintain continuous culture of the organoids, passage expansion may be carried out just prior to epithelial shedding depending on the cell lines utilized.

In some embodiments, Caco-2 cell lines are used for drug oral absorption assays. Caco-2 cell lines express efflux and uptake transporters found in the human intestinal tract. Caco-2 cells may also be used as models to determine if a drug is a substrate or inhibitor of efflux transporters. Although the Caco-2 cells are of colonic origin, they mimic an enterocyte cell. Notably, goblet cells, dedicated to mucus production, are absent from Caco-2 cultures. In embodiments, the 3D intestinal organoid technology described herein is used to supplement Caco-2 cell lines. Specifically, 3D canine organoids provide an in vitro system for exploring canine drug permeability, metabolism, active transport, and drug-drug interactions. For example, a permeability assay with canine intestinal organoids may predict canine intestinal permeability and metabolism of small drug molecules compared to currently used assays (Caco-2). In embodiments, said organoids may be used to assess the impact of inducers on intracellular metabolic and on active transport.

Further to the above, in some embodiments said differentiation media, Caco-2 cell lines, and/or 3D canine organoids may be used in combination with transwell systems. In embodiments, said transwell systems may be used to determine the apparent permeability of therapeutic drug candidates. Said transwell systems can also be employed to assess cellular secretion, cell migration, and drug toxicity.

In one embodiment of a transwell system, a dual-chamber cell culture apparatus comprised of an insert with a semiporous membrane is placed in a multiwell plate. This system allows direct access to the apical and basolateral sides of a cell-monolayer grown on the insert. The monolayer used in this system may be derived from hepatic cells. Specifically, cell cultures are grown in a polarized state mimicking the natural microarchitecture of hepatic cells, enabling cellular differentiation, microanatomy, and function. The seeding of the inserts with 2D cell cultures has been traditionally used for assessing drug oral permeability, is relatively affordable, and is easy to culture.

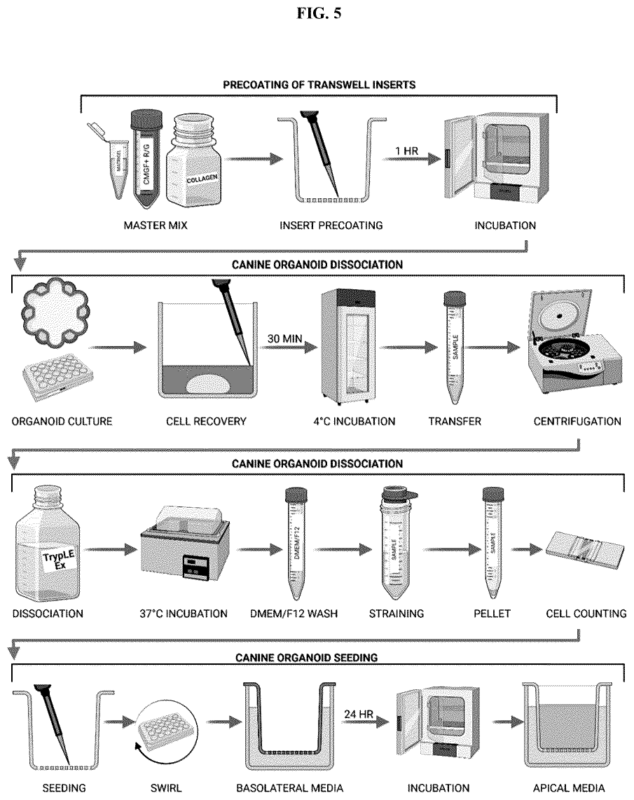

As depicted in FIG. 5, in one example, a transwell seeding protocol (TSP) includes a precoating of transwell inserts, canine organoid dissociation, canine organoid seeding, TEER Value Measurement, and monolayer upkeep. An exemplar workflow for TSP and a permeability experimental protocol is provided in Example 3 and Example 4 below. Said examples elaborate on the steps of evaluating organoid monolayer readiness, preparing for the experiment, typical experimental layout, organoid monolayer quality control, and fixing cell monolayers for downstream analysis. As disclosed is the procedure for pre-coating of the inserts with collagen I and Matrigel. Embedding of canine organoids on the Transwell inserts is also disclosed.

Further to the above, a second disclosed protocol (referred to as a “monolayer maintenance protocol”) is provided in the Examples section. Said protocol includes methods for general upkeep of canine 3D organoids plated on an insert. The frequency and volumes of the organoid media used to refresh the culture, and ways to prevent cell culture damage, are presented in this second protocol along with experimental methods for assessing the confluency of the epithelial monolayer.

In additional embodiments, a “permeability experimental protocol” is provided that focuses on ways to determine if the canine hepatic 3D organoid on a Transwell assay is ready for experimental use and the verification steps needed prior to conducting any experiment. This section also describes the set up and the successful execution of a permeability experiment, along with the incubation and sampling of therapeutic drug candidates in the chambers of the monolayer culture. Also disclosed are uses of the low permeability fluorescein isothiocyanate (FITC-dextran) as a means to monitor monolayer integrity. In other embodiments, disclosed are an in vitro evaluation method for validating the results after the conclusion of an experiment.

As described in further detail in the Examples, in one example a Transwell insert is pre-coated with a mixture of Matrigel and collagen I and incubated for 1 hour. During the incubation process, the organoid culture is dissociated. Individual organoid cells are seeded in the insert, and media is added to the apical chamber 24 hours after the seeding process concludes. The organoid culture may be cultivated for at least four days without any disturbance. Maintenance and monitoring of the organoids include regular media changes, TEER value measurements, and light microscopy to evaluate the integrity of the monolayer. Before the experiment, the organoids may be differentiated by removing Rock inhibitor and GSKir3 from the media. The TEER values are measured on the experiment day, and the organoid monolayer is inspected via light microscopy for damage to the cells. Media is then exchanged for an appropriate buffer and incubated prior to the experiment. The FITC-dextran assay is used during hepatic permeability experiments as a marker of monolayer integrity, TEER measurements are taken after the experiment and light microscopy will validate the results after 24 hours.

### Organoid Media

The organoids may be grown in any acceptable media. In an embodiment, the cells may be grown in a basal media, such as but not limited to DMEM, GIBCO™ ADVANCED™ DMEM, MEM, RPMI 1640, Opti-MEM, McCoy's 5A, Hybri-Care, Leibovitz's L-15, or IMEM. The basal media may further be supplemented with nutrient mixes, such as, but not limited to F-12 and/or F-10, L-glutamine, fetal bovine serum (FBS), growth factors, additional salts, pathway inhibitors, antimicrobials, additional buffers, and/or other additives, and/or mixtures thereof to make a more complete media. Antimicrobials may include any cell culture grade antibiotics and/or antifungals. In a preferable embodiment, the media is a complete media and comprises of the basal media DMEM and is supplemented with F-12, L-glutamine, HEPES buffer, and PRIMOCIN™, available from InvivoGen (Complete Media without Growth Factors, CMGF- media), even more preferably, DMEM/F-12 supplemented with about 1 mM to about 2 mM GlutaMax-1 as an L-glutamine source, from about 5 mM to about 15 mM HEPES, and from about 80 μg/mL to 100 μg/mL PRIMOCIN™. The supplements may be added to the basal media prior to contact with the organoids or the supplements may be added after the organoids are in culture. A variety of organoid media and Formalin-Acetic Acid-Alcohol (FAA) solutions are disclosed in Example 2 below.

In another embodiment, the media is a differentiation media. In a preferred embodiment, the differentiation media includes a complete media supplemented with growth factors and/or other supplements. In a particularly preferred embodiment for epithelial organoids, the growth factors and supplements include B27 (available from Thermo Fisher Scientific), N2 (available from Thermo Fisher Scientific), epidermal growth factor (EGF), Noggin, a transforming growth factor beta receptor I inhibitor (TGFβ type I), a mitogen activated protein kinase 14 (P38) inhibitor, DAPT, dexamethasone, and FBS. Surprisingly, the inclusion of R-Spondin-1, wingless-type MMTV integration site family member 3A (Wnt3a), Gastrin, and Nicotinamide prevented the differentiation of the hepatic organoids, and the media is preferably substantially free of Wnt3a, Gastrin, R-Spondin-1, and Nicotinamide.

In a more preferred embodiment, the differentiation media is Complete Media with Growth Factors (CMGF+) supplemented with 1× B27 (Fisher), 1× N2 (Fisher), from about 250 mM to about 750 mM N-acetylcysteine, from about 40 ng/ml to about 60 ng/mL EGF, from about 80 ng/mL to about 120 ng/mL Noggin, from about 250 nM to about 600 nM A83-01 (TGFβ type I receptor inhibitor), from about 5 μM to about 15 μM SB202190 (P38 inhibitor), from about 6 μM to about 14 μM DAPT, from about 1 μM to about 5 μM dexamethasone, and from about 6% to about 10% FBS. The media may include or be substantially free or free from other, known growth factors, such as but not limited to angiopoietin (ANG), bone morphogenic proteins (BMP), colony-stimulating factor (CSF), erythropoietin (EPO), fibroblast growth factor (FGF), insulin, migration-stimulating factor (MSF), myostatin (GDF-8), neuregulins, neurotrophins, interleukins, R-Spondin-1, Wnt3a, Gastrin, Nicotinamide, and/or placental growth factor (PGF).

In a different embodiment, the media is a protecting media. In a preferred embodiment, the protecting media is a complete media with the addition of a rho kinase (ROCK) and/or glycogen synthase kinase 3 (GSK-3) inhibitor. Rho kinase inhibitors include, but are not limited to Y27632, Y39983, Wf-536, SLx-2119, Azabenzimidazole-aminofurazans, DE-104, olefins, isoquinolines, indazoles, pyridinealkene derivatives, H-1152P, ROKa inhibitors, XD-4000, HMN-1152, 4-(1-aminoalkyl)-N-(4-pyridyl)cyclohexane-carboxamides, Rhostatin, BA-210, BA-207, BA-215, BA-285, BA-1037, Ki-23095, VAS-012, and fasudil. Many GSK-3 inhibitors are known in the art, the GSK-3 inhibitor is preferably an aminopyrimidine, and more preferably CHIR99021. In a preferred embodiment, the protecting media includes ROCK and GSK-3 inhibitors in CMGF+. In a more preferred embodiment, the protecting media includes from about 8 μM to about 12 μM ROCKi and from about 1.5 μM to about 3.5 μM CHIR99021. Without being bound to a particular theory it is believed that the addition of the inhibitors may enhance the initial survival and facilitate long-term propagation of endothelial organoids if included in the initial culture. It is believed that the inhibitors take the place of Paneth cells in canines for early differentiation of the stem cells into organoids.

In yet another embodiment, the media is a “freezing media”. For example, commercial media like Recovery™ cell freezing media may be used. It has been surprisingly found that when the cells are frozen in a media comprising from about 40% to about 60% v/v CMGF+, from about 30% to about 50% v/v FBS, and from about 5% to about 15% v/v dimethyl sulfoxide (DMSO) not only the amount of time needed for cells to grow is decreased, but more are recovered when compared to commercial media.

### Two-Dimensional Membrane Models

In an embodiment, after the organoids have formed, they may be further used to make two-dimensional (2D) membrane models. This may allow easier access to the lumen or to expose each side of the organoid to a different media or environments. The organoids are first lysed into a single cell mix. Lysis may be achieved by either mechanically or chemically disrupting the organoids, such as mechanical pipetting or using trypsin. The single cell mix is then diluted to a concentration of about 1×10³ cells/mL 1×10⁴ cells/mL, about 1×10⁵ cells/mL, about 1×10⁶ cells/mL, or about 1×10⁷ cells/mL. An appropriate number of cells are then transferred onto a membrane, preferably a permeable membrane, or into a well of a TRANSWELL® plate. The cells may be transferred and cultured in an appropriate extracellular matrix for about 1 hour, for about 2 hours, or for about 3 hours. The cells are then washed and cultured for about 8 to about 16 days, from about 10 to about 14 days, or from about 12 to 13 days.

In one embodiment, a method of making a P-gp model further comprises lysing a hepatic organoid into single cells; transferring into a TRANSWELL® well; and culturing.

In an embodiment, the membranes are permeable. In a further embodiment, the membrane may be part of a microfluidics system. In an embodiment, the microfluidics system has a single chamber for the introduction of media to one side of the membrane. In another embodiment, the microfluidics system has two chambers for media on either side of the membrane allowing media to be introduced to both sides of the membrane. In an embodiment, the two chambers are filled with the same media. In another embodiment, each chamber is filled with different media.

### Methods for Passaging, Freezing, and Recovering Organoids

While any acceptable passaging, freezing, or recovery protocol may be used for the organoids, it has been surprising found that certain methods and compositions increase cell yield and growth efficacy of the organoids. The methods presented are for 24 well culture plates. One skilled in the art will appreciate that the volumes and cell densities involved will change depending on the size of the culture plate being used and can scale up or down as necessary.

### Organoid Passage

Usually after about 4 to about 7 days, the organoids are ready to be passaged. A first exemplary method of passaging and cleaning the cells for a single well of a 24 well culture plate comprises:
1. Remove medium from wells (around the solid extracellular matrix) with, for example, a P1000 pipet, 5 ml pipet, or Pasteur pipet/aspirator vacuum.
2. Add about 300 μl to about 800 μl, from about 400 μl to about 700 μl, from about 450 μl to about 550 μl cold complete media, such as, but not limited to, CMGF− or DMEM/F12, to the well and mechanically break up the extracellular matrix with pipetting, preferably with a large pipette, such as a P1000 or P5000, by pipetting up and down a sufficient number of times, for example 3 or 4 times.
3. Transfer organoids and media to a centrifuge tube, preferably a 15 ml conical tube.
4. Spin down in refrigerated centrifuge sufficiently to pellet the cells, for example at about 100 g for about 5 min at about 4° C.
5. Remove the supernatant and resuspend pellet in about 0.7 ml to about 1.5 ml protease solution, preferably trypsin or TrypLE Express, and put tube in 37° C. water bath for about 7 to about 10 minutes.
6. Add about 4 ml to about 5 ml complete media, by way of nonlimiting example DMEM/F12 or CMGF-, to stop dissociation of cells.
7. Spin down in a centrifuge to pellet the cells, for example at 100 g for 5 min at 4° C.
8. Remove the supernatant though aspiration, for example by using a 5 ml or 10 ml pipet, then P1000 or P200 pipet or an aspirator to pull the media off the pellet. Keep tube on ice.
9. Resuspend organoid pellet in an extracellular matrix (calculate the amount of extracellular matrix you will need, preferably about 25 μl/well to about 30 μl/well) using cold pipet.
10. Pipet designated amount of μl/well, for a well on a 24 well plate, it is preferable to use from about 25 μl to about 30 μl /well) of organoid/extracellular matrix mixture as a droplet using a P20, P100, or P200 cold pipet tip. Transfer plate/dish into 37° C. 5% CO₂ incubator. Let matrix settle for about 5 to about 20 minutes, add about 300 μl to about 800 μl of room temperature differentiation media, preferably CMGF+, to each well and culture in 37° C. incubator. Optionally, may use a conditioned differentiation media that is about 40% to about 60% conditioned medium (CM from WRN cells) and about 40% to about 60% differentiation media, may need to sterile filter media. Can either put in same number of wells or split 1:2 to 1:4, depending on organoid density.
11. Refresh culture with differentiation media as needed, preferably every other day.

As shown in FIG. 5, a second exemplary method, referred to as “the Transwell Seeding Protocol” (TSP), comprises:
1. Transwell inserts are pre-coated with a combination of CMGF+ RIG, collagen I, and Matrigel, and subsequently incubated.
2. Media from the canine organoid culture is aspirated and replaced with a Cell Recovery Solution, followed by a 30 min incubation at 4° C.
3. The culture is subsequently transferred to a tube, and organoid dissociation is performed using TrypLE Express.
4. Undissociated organoids are removed by passage through a strainer to achieve a single cell suspension, and cell concentration is determined using a hemocytometer or an automated cell counter.
5. The cells are seeded on a Transwell insert, and CMGF+ R/G is added to the basolateral chamber.
6. The culture is then incubated for 24 hr, and the remaining liquid is removed from the apical chamber and replaced with CMGF+ RIG.

Notably, the TSP method is disclosed in greater detail in the Examples section below.

### Clean Up Organoids

After about 2 to about 4 days, organoids passaged with a protease, such as trypsin or TrypLE, may need to be cleaned up to remove debris, dead cells, and single cells (usually differentiated cells). To clean the cells, follow the steps to passage the organoids as above, omitting steps #6-8. The organoids may either be put in same number of wells or split 1:2 to 1:4, depending on organoid density.

### Organoid Freezing Protocol

Any freezing media may be used to freeze the cells using methods known in art. However, it has been surprisingly found that the freezing media described in the Organoid Media section increases cell viability. If using a 24 well culture plate, it is preferable to increase the cell concentration in a cryovial by combining two or more wells. Usually after about 2 or 3 days after passaging as described above (unless they need clean-up to remove debris), organoids may be frozen. A preferable, exemplary method for 24 well plates of freezing cells to improve recovery comprises:
1. Remove medium from wells (around the solid extracellular matrix) using, for example, a P1000 pipet, 5 ml pipet or Pasteur pipet/vacuum aspirator.
2. Add about 300 μl to about 800 μl cold complete media, preferably CMGF− or DMEM/F12, to well and mechanically break up the extracellular matrix, preferably by pipetting up and down a sufficient number of times.
3. Spin down in refrigerated swing rotor centrifuge to pellet the cells, for example at 100 g for 5 min at 4° C.
4. Remove all medium. Keep tube on ice.
5. Resuspend hepatic organoids into freezing medium (using about 300 μl to about 800 μl for each cryovial) at original ratio of 2 wells into 1 vial, if using a 24 well plate.
6. As an optional step, before placing into liquid nitrogen, the cryovial may be kept at below about −76° C. in a freezer, preferably in a −80° C. freezer, for up to 1 week. Then transfer vials into liquid nitrogen for long-term storage.

### Organoid Revival Protocol

Any method may be used to revive (thaw) organoids from liquid nitrogen. However, it has been surprisingly found that the number of cells recovered and the amount of time it takes to grow the organoids may be improved by:
1. Thaw an extracellular matrix aliquot on ice in black anodized aluminum cooling block and pre-warm plate
2. Add about 8 ml to about 10 ml of a complete media, such as, but not limited to, CMGF− or DMEM/F12, into a 15 ml tube, leave tube on ice
3. Optionally, transfer frozen vial containing organoids from liquid nitrogen to dry ice
4. Swirl vial in about 37° C. water until thawed (liquid)
5. Immediately transfer contents in the vial to 15 ml tube containing about 10 ml cold complete media drop by drop
6. Spin down a centrifuge at 100 g for 5 min at 4° C.
7. Remove medium and leave 15 ml tube containing organoid pellet on ice. Resuspend pellet in about 60 μl to about 120 μl of extracellular matrix (enough to seed about 4 wells with 15 μl to about 30 μl/well of matrix) using a cold pipet tip, plate organoids as droplets in 4 wells of 24 well plate and transfer plate to 37° C. incubator.
8. Let gel settle for 5-20 minutes, add about 300 μl to about 800 μl of room temperature protecting media to each well and culture in 37° C. incubator.
9. Refresh culture with differentiation media as needed, preferably every other day, until ready to be passaged (typically 5-7 days).

### Models and Methods of Use

### Methods for Use in Diseases

The organoid models described above may be used in detecting differences in organoids due to disease by detecting changes in RNA or protein expression or detecting changes in concentrations of metabolites within the organoids or within the culture media. For example, tissue samples may be taken from a diseased subject and differences in RNA or protein production may be detected in comparison to a control subject lacking the disease. Alternatively, both the diseased and control samples may be derived from the same subject to detect within subject differences. Detecting difference from the within subject comparison may show how the disease developed locally more clearly than an across subject comparison.

Alternatively, genetically modified organoids may be used to determine the role of genes which may be the cause of the disease, or which may provide resistance to a disease. For example, if a knockout of a protein, such as, but not limited to, a transcription factor or DNA repair gene results in immortalization or tumor development in a healthy sample, it may be concluded that that protein is a proto-oncogene in the hepatic tissue.

In an embodiment, the organoids and methods of use provide an effective model for identifying differences from human models and animal models, preferably for canine species. This is particularly important when differences between humans and canines emerge. The organoids and methods of use provide an ex vivo model for use in canine species. The three-dimensional culture conditions provide effective tools for modeling healthy and diseased subject response to a drug or an environmental or dietary trigger.

In an embodiment, to determine if there is an age difference in the response to a trigger, serial samples of stem cells may be taken from the same subject to produce longitudinal studies. Some carnivores, such as canines, due to their shorter lifespan but similar habitual diets compared to humans, may beneficially provide a more rapid development of a model for chronic diseases which may how an environmental or dietary trigger interacts in vivo over time.

The three-dimensional culture conditions provide a platform for modeling various phenotypes, associated with a subject-specific trait or mutation. This can be useful in gene editing studies that confirm subject-specific variations in genetic and epigenetic changes that may benefit from personalized therapies and/or administration of therapies on a personalized basis.

### Methods for Use in Environment Responses

The organoid models described above may be used in detecting changes to the organoids due to environmental changes by detecting changes in RNA or protein expression, changes in epigenetics, such as DNA methylation or histone modifications, or detecting changes in concentrations of environmental factors or their metabolites. For example, an environmental or dietary trigger may be added to the media and the hepatic organoids may be used to measure the transport and metabolism of the trigger from surrounding media to estimate the apparent permeability and hepatic metabolism of the trigger. The trigger may be any environmental or dietary trigger, such as, but not limited to, pathogens or their components, such as whole bacteria, viruses, or paramecium or components such as lipopolysaccharide or viral proteins; heavy metals; chemicals, such as volatile organic compounds, phthalates, or formaldehyde; or small molecules, such as carbon monoxide, arsenic, or cyanide.

Alternatively, genetically modified organoids may be used to determine the role of genes which may be responsible for the uptake or metabolism of environmental or dietary trigger. For example, if a knockout of a transporter protein reduces the removal of the trigger from solution while overexpression increases removal, then it may be concluded that that transporter may at least partially transport the trigger, depending on the change in removal.

The organoids may be used by measuring the rate or amount of trigger may be taken into the interior of the organoid. Similarly, an organoid cultured on a permeable membrane, such as a TRANSWELL® plate, may be used to measure transfer of the trigger across the membrane.

In other embodiments, a trigger may be added to the culture media and then the media sampled to detect changes in compounds known to be produced by the organoids. This detection may show what downstream effect the trigger has on the hepatic tissue from which the organoid derives.

In an embodiment, the organoids and methods of use provide an effective model for identifying differences from human models and animal models, preferably for canine species. This is particularly important when differences between humans and canines emerge. The organoids and methods of use provide an ex vivo model for use in canine species. The three-dimensional culture conditions provide effective tools for modeling healthy and diseased subject response to a trigger.

In an embodiment, to determine if there is an age difference in the response to a trigger, serial samples of stem cells may be taken from the same subject to produce longitudinal studies. Canine, due to their shorter lifespan but similar diets compared to humans, may beneficially provide a more rapid development of a model for chronic diseases which may be used to investigate how a trigger interacts in vivo over time.

The three-dimensional culture conditions provide a platform for modeling various phenotypes, associated with a subject-specific trait or mutation. This can be useful in gene editing studies that confirm subject-specific variations in genetic and epigenetic changes that may benefit from personalized therapies and/or administration of therapies on a personalized basis.

### Methods for Use in Diet and Diet Changes

The organoid models described above may be used in detecting changes to the organoids due to changes in diet or additives to a diet by detecting changes in RNA or protein expression, changes in epigenetics, such as DNA methylation or histone modifications, or detecting changes in concentrations of metabolites. For example, an initial diet may be provided to the organoid followed by removal of a compound or the addition of a compound. Change in gene or protein expression or the concentrations of metabolites within the cells or media may then be detected. A detected change may allow for measuring the effects a change in diet has on energy levels or toxicity of a given diet or additive.

Alternatively, genetically modified organoids may be used to determine the role of genes which may be responsible for the uptake or metabolism of dietary compounds. For example, if a knockout of a transporter protein reduces the removal of a compound found within the diet from solution while overexpression increases removal, then it may be concluded that that transporter may at least partially transport the dietary compound, depending on the change in removal.

The organoids may be used by measuring the rate or amount of the diet or a component thereof may be taken into the interior of the organoid. Similarly, an organoid cultured on a permeable membrane, such as a TRANSWELL® plate, may be used to measure transfer of the dietary compound across the membrane.

In an embodiment, the organoids and methods of use provide an effective model for identifying differences from human models and animal models, preferably for canine species. This is particularly important when differences between humans and canines emerge. The organoids and methods of use provide an ex vivo model for use in canine species. The three-dimensional culture conditions provide effective tools for modeling healthy and diseased subject response to a diet or a change in diet.

In an embodiment, to determine if there is an age difference in the response or ability to metabolize to a given diet or a compound within the diet, serial samples of stem cells may be taken from the same subject to produce longitudinal studies. Canine, due to their shorter lifespan but similar diets compared to humans, may beneficially provide a more rapid development of a model for how a subject is capable of metabolizing a diet over time.

The three-dimensional culture conditions provide a platform for modeling various phenotypes, associated with a subject-specific trait or mutation. This can be useful in gene editing studies that confirm subject-specific variations in genetic and epigenetic changes that may benefit from personalized therapies and/or administration of therapies on a personalized basis.

### Models for P-Glycoprotein-Mediated Drug Transport

The above organoids may be used to make diverse models, which can be used for assaying P-gp mediated drug transport.

In some embodiments, the model for P-gp transport comprise hepatic organoids, wherein the organoids are differentiated from Lgr5-positive stem cells. In preferred embodiments, the Lgr5-positive stem cells are obtained from canines.

In some embodiments the organoids express wild-type levels P-gp. In other embodiments the organoids have been genetically modified to alter the expression of P-gp. In some embodiments, the genetic modification knockdowns, knockouts, or overexpresses P-gp.

In further embodiments, the organoids are cultured in a monolayer on a TRANSWELL® membrane. In an embodiment, the TRANSWELL® membrane is permeable.

In some embodiments, the models include a P-gp inhibitor. P-gp inhibitors include, but are not limited to, amiodarone, clarithromycin, ciclosporin, colchicine, diltiazem, erythromycin, felodipine, ketoconazole, lansoprazole, omeprazole and other proton-pump inhibitors, nifedipine, paroxetine, reserpine, saquinavir, sertraline, quinidine, tamoxifen, verapamil, duloxetine, elacridar, CP 100356, zosuquidar, tariquidar, valspodar and reversan.

In other embodiments, the models include a P-gp inducer. P-gp inducers include, but are not limited to, carbamazepine, dexamethasone, doxorubicin, nefazodone, phenobarbital, phenytoin, prazosin, rifampicin, St. John's wort, tenofovir, tipranavir, trazodone, and vinblastine.

In yet other embodiments, the models include a P-gp substrate. Substrates of P-gp are susceptible to changes in pharmacokinetics due to drug interactions with P-gp inhibitors or inducers. Some of these substrates include colchicine, ciclosporin, dabigatran, digoxin, diltiazem, fexofenadine, indinavir, morphine, and sirolimus.

### Methods of Making P-Glycoprotein Models

Traditional 2D cell cultures involving immortalized cells, such as cancer cells with or without a genetic modification to express specific proteins, such as, but not limited to, P-gp, or primary cells have been used in coverslip or standard wells. However, the absence of a basolateral compartment precludes cell polarization and may prevent the study of transport across cell layers. Further, the use of cancer cells, or other immortalized cells, or genetic modification may lead to changes in expression of protein when compared to the normal physiological state. Therefore, 3D models using cells differentiated from initial stem cells may result in models which are more like the normal physiological state than 2D models. Such models include, but are not limited to, 3D organoids and TRANSWELL® cultures.

In an embodiment, the organoid models may be used to study P-gp and drug permeability, efficacy, and safety. For P-gp models, a sample of organoids may be taken and the expression and/or localization of P-gp nucleic acid or protein may be assayed. In some embodiments, PCR may be used to detect the expression of P-gp RNA. In other embodiments, immunohistochemistry (IHC) may be used to measure the expression and/or localization of P-gp protein. Western blots may also be used to quantify the amount of P-gp protein. If the organoids have been genetically modified, then quantitative PCR or IHC/immunofluorescence may be used to quantify the change in expression of P-gp.

In an embodiment, a method of making a P-gp model comprises obtaining an hepatic sample; extracting leucine-rich repeat-containing G protein-coupled receptor 5 (Lgr5)-positive stem cells; culturing said Lgr5-positive stem cells within an extracellular matrix, wherein the culture media causes differentiation of the stem cells; maintaining the culture until organoids form, wherein the organoids are positive for P-gp expression.

In further embodiments, the stem cells are genetically modified. In some embodiments the genetic modification knockdowns the expression of P-gp. In other embodiments the genetic modification overexpressed P-gp. In yet other embodiments the genetic modification alters the cellular location of P-gp. In yet another embodiment, the genetic modification mimics mutation in a disease.

In a preferred embodiment, the hepatic sample is obtained from a canine subject.

In an embodiment, the hepatic sample is from the liver. In another embodiment, the hepatic sample is from the bile duct.

### Use of Models in Drug Development and Screening

The above compositions may be used in drug development and screening by measuring transport (i.e., drug efflux) through transporters, such as, but not limited to, P-gp. For example, the hepatic organoids may be used to measure the hepatic transport and metabolism of a compound from surrounding media to estimate the apparent permeability and hepatic metabolism of the compound. The compound may be a drug or a P-gp substrate. If the compound is a test drug, then a P-gp inhibitor or inducer may be co-administered with the drug to determine if P-gp transports the drug out of solution by measuring an increase or decrease in drug permeability, respectively. Since P-gp is an efflux protein, inhibiting P-gp-mediated drug transport will result in an increase in drug permeability from the donor i.e., apical to the receiver i.e., basal side of the TRANSWELL®. Alternatively, genetically modified organoids may be used to determine the role of P-gp on said drugs removal. For example, if a knockout reduces the removal from solution while overexpression increases removal, then it may be concluded that P-gp may at least partially transport the drug, depending on the change in removal.

If the compound is a P-gp substrate, then a drug may be co-administered with the substrate in order to determine which of the drugs may interfere with P-gp mediated transport of the substrate out of solution by observing a change in the rate of removal from solution.

The organoids may be used to estimate hepatic permeability by measuring the rate or amount of substrate or drug taken into the interior of the organoid. Similarly, an organoid cultured on a permeable membrane, such as a TRANSWELL® plate, may be used to measure transfer of the drug across the membrane.

In an embodiment, the organoids and methods of use can be used to assess a variety of therapeutic drugs. In an embodiment, exemplary therapeutic drugs include, nonsteroidal anti-inflammatory drugs (NSAIDs), chemotherapy drugs, etc. Any candidate drug may be tested, for example the drug molecules from the Biopharmaceutics Classification System (BCS). See Amidon GL, et al., 1995, A Theoretical Basis For a Biopharmaceutics Drug Classification: The Correlation of In Vitro Drug Product Dissolution and In Vivo Bioavailability, Pharm Res, 12: 413-420. The BCS is a scientific framework for classifying drug substances based on their aqueous solubility and hepatic permeability. When combined with the dissolution of the drug product, the BCS takes into account three major factors that govern the rate and extent of drug absorption from IR solid oral dosage forms: (1) dissolution, (2) solubility, and (3) hepatic permeability.

According to the BCS, drug substances are classified as follows:
1. Class 1: High Solubility—High Permeability
2. Class 2: Low Solubility—High Permeability
3. Class 3: High Solubility—Low Permeability
4. Class 4: Low Solubility—Low Permeability.

In an exemplary embodiment, the organoids and methods of use described herein can provide effective models to assess therapeutic efficacy of such exemplary therapeutic drugs including, nonsteroidal anti-inflammatory drugs (NSAIDs), chemotherapy drugs, etc. In a further embodiment the organoids and methods of use described herein can assess therapeutic failures and toxicity, including exposure-associated toxicity, of such exemplary therapeutic drugs including, nonsteroidal anti-inflammatory drugs (NSAIDs), chemotherapy drugs, etc. In still further embodiments, the organoids and methods of use described herein can assess how the exemplary therapeutic drugs will affect the liver of a subject, providing ability to determine any rate limiting dosages of the therapeutic drugs.

In an embodiment, the organoids and methods of use provide an effective model for identifying differences from human models and animal models, namely for canine species. This is particularly important when differences between humans and canines emerge. The organoids and methods of use provide an ex vivo model for use in canine species. The three-dimensional culture conditions provide effective tools for modeling healthy and diseased subject oral absorption and/or elimination of drugs.

In an embodiment, serial samples of stem cells may be taken from the same subject to produce longitudinal studies. Canine, due to their shorter lifespan but similar diets compared to humans, may beneficially provide a more rapid development of a model for chronic diseases which may how a drug interacts in vivo over time.

In an embodiment, canine hepatic stem cells taken from healthy dogs provide an accurate predictor of the efficacy of the therapeutic drugs being tested as they closely mimic biological responses and physiologic state in dogs, providing a good predictor of therapeutic efficacy in vivo based on cells produced in vitro. In a still further embodiment, canine hepatic stem cells taken from diseased dogs better predict the efficacy of the therapeutic drugs being tested and more closely mimic biological responses and the physiological state in such diseased dogs, providing a good predictor of therapeutic efficacy in vivo based on cells produced in vitro. Such methods of screening of potential therapeutic drugs and screening of drug responses in ex vivo models beneficially speed up the drug testing timeline to trials as well as provide a better predictor of efficacy in the canines with similar diseases to the animals that the canine cells were taken from for producing the organoids.

The three-dimensional culture conditions provide a platform for modeling various phenotypes, associated with a subject-specific trait or mutation. This can be useful in gene editing studies that confirm subject-specific variations in genetic and epigenetic changes that may benefit from personalized therapies and/or administration of therapies on a personalized basis.

### Differentiation of Hepatic Organoids

As shown in FIGS. 1A-D, immunohistochemistry (IHC) shows LGRS, a marker for undifferentiated stem cells, having strong staining (blue) up to about day 6, but began to decrease on day 7. IHC for KRT7, a marker for cholangiocytes, also shows strong expression starting on day 2 and persisting through day 11 (see FIGS. 2A-C). FIGS. 3A-B and 4A-B show IHC staining for CYP3A12 and CYPA4, markers for mature hepatocyte cells. As shown, mature hepatocytes appeared by day 7 and persisted to day 11.

As described in the Examples below, RNAscope® 2.5 was performed for KRT7 and AQP1 as markers for cholangiocytes, LGR5 as a marker for stem cells, and FOXA1 as a marker for early hepatocytes and CYP3A12 for mature hepatocytes. As shown in Table 1, all cell types were obtained over the initial 7-day cultures in varying amounts.

TABLE 1

| Spheroids | | in total area signal/total area cell | | | | | |
|---|---|---|---|---|---|---|---|
| CELL TYPE | MARKER | DAY2 | DAY3 | DAY4 | DAY5 | DAY6 | DAY7 |
| CHOLANGIOCYTE | KRT-7(shorter) | 26.02% | 1.00% | 9.26% | 2.16% | 7.63% | 15.08% |
| STEM CELL | LGR5(longer) | 0.78% | 0.43% | 0.17% | 0.69% | 0.26% | 0.39% |
| CHOLANGIOCYTE | AQP1 | 0.00% | 0.00% | 0.00% | 0.00% | 0.00% | 0.01% |
| HEPATOCYTE-EARLY | FOXA1 | 0.16% | 0.34% | 0.07% | 0.07% | 0.05% | 0.20% |
| HEPATOCYTE-MATURE | CYP3A12 | 0.28% | 0.05% | 0.11% | 0.05% | 0.04% | 0.03% |

The results shown in Table 1 demonstrate that a variety of hepatic organoids may be obtained using the differentiation CMGF+ media and methods disclosed herein. In embodiments, this will allow for a variety of tests or assays to be performed on both hepatic cells and hepatic derived epithelial cells from cholangiocytes.

Further to the above, one skilled in the art will appreciate that even more complex experimental designs are possible with the organoids. For example, the interaction between diet, treatment, and disease may be determined by combining the methods relating to each design. More specifically, a nonlimiting example of a more complex design may be to detect cell viability between a healthy population of organoids receiving a specified diet, a diseased population of organoids receiving the same specified diet, a healthy population of organoids receiving a higher protein or fat diet, and a diseased population of organoids receiving the same higher protein or fat diet. Additionally, the organoids could further be treated with, for example, a chemotherapy regime if the disease is cancer. This may allow one to determine if there are any interaction effects among diet, disease, and treatment. Further considerations may also include longitudinal studies as described above to determine if age may play a role in any interaction effects.

In summary, the herein described compositions include models for the study of developmental biology of the liver and other epithelial tissues, drug discovery and toxicity screening, infectious disease biology of various infectious agents, and personalized medicines. In other embodiments, methods for seeding canine intestinal organoids, maintaining an organoid monolayer, and monitoring monolayer integrity are provided. As described below, methods and systems for culturing, freezing, and recovering the frozen organoid cells are also provided. Finally, the hepatic organoids may also be used to treat a subject in need, or for identifying a preferred therapeutic agent.

In embodiments, the organoids are used to treat cancer, a cancer cell, or cancer tissue. The cancer cell may be an epithelial, an endothelial, a mesothelial, a glial, a stromal, or a mucosal cell. The cancer cell population can include, but is not limited to a brain, a neuronal, a blood, an endometrial, a meninges, an esophageal, a lung, a cardiovascular, a liver, a lymphoid, a breast, a bone, a connective tissue, a fat, a retinal, a thyroid, a glandular, an adrenal, a pancreatic, a stomach, an intestinal, a kidney, a bladder, a colon, a prostate, a uterine, an ovarian, a cervical, a testicular, a splenic, a skin, a smooth muscle, a cardiac muscle, or a striated muscle cell. In still a further aspect cancer includes, but is not limited to astrocytoma, acute myeloid leukemia, anaplastic large cell lymphoma, acute lymphoblastic leukemia, angiosarcoma, B-cell lymphoma, Burkitt's lymphoma, breast carcinoma, bladder carcinoma, carcinoma of the head and neck, cervical carcinoma, chronic lymphoblastic leukemia, chronic myeloid leukemia, colorectal carcinoma, endometrial carcinoma, esophageal squamous cell carcinoma, Ewing's sarcoma, fibrosarcoma, glioma, glioblastoma, gastrinoma, gastric carcinoma, hepatoblastoma, hepatocellular carcinoma, Kaposi's sarcoma, Hodgkin lymphoma, laryngeal squamous cell carcinoma, larynx carcinoma, leukemia, leiomyosarcoma, lipoma, liposarcoma, melanoma, mantle cell lymphoma, medulloblastoma, mesothelioma, myxofibrosarcoma, myeloid leukemia, mucosa-associated lymphoid tissue B cell lymphoma, multiple myeloma, high-risk myelodysplastic syndrome, nasopharyngeal carcinoma, neuroblastoma, neurofibroma, high-grade non-Hodgkin lymphoma, non-Hodgkin lymphoma, lung carcinoma, non-small cell lung carcinoma, ovarian carcinoma, esophageal carcinoma, osteosarcoma, pancreatic carcinoma, pheochromocytoma, prostate carcinoma, renal cell carcinoma, retinoblastoma, rhabdomyosarcoma, salivary gland tumor, Schwannoma, small cell lung cancer, squamous cell carcinoma of the head and neck, testicular tumor, thyroid carcinoma, urothelial carcinoma, and Wilm's tumor.

### EMBODIMENTS

Various embodiments of the herein disclosed organoids and methods provided herein are included in the following non-limiting list of embodiments, referred to as paragraph 1 to 88 below.
1. A canine hepatic organoid, comprising a population of differentiated canine hepatic-derived cells which are capable of organ-like functionality, including production of bile-related compounds, production of blood plasma proteins, production of cholesterol, conversion of glucose into glycogen, hemoglobin processing, production of immune factors, and the like; and optionally wherein the hepatic organoid is genetically modified to alter the expression of P-glycoprotein (P-gp).
2. The hepatic organoid of paragraph 1, wherein the hepatic-derived cells are adult stem-cell derived cells, adult stem-cell-derived organoids, and the like.
3. The hepatic organoid of paragraph 1, wherein the hepatic-derived cells are induced pluripotent derived stem cells.
4. The hepatic organoid of paragraph 1, further comprising an extracellular matrix, wherein the hepatic organoid maintains the organ's three-dimensional structures, extracellular macromolecules and minerals (e.g., such as collagen, enzymes, glycoproteins and hydroxyapatite) that provide structural and biochemical support to surrounding cells.
5. The hepatic organoid of paragraph 4, wherein the hepatic-derived cells are epithelial cells and maintain the expression of tight junction proteins, such as occludin, claudin and junctional adhesion molecules.
6. The hepatic organoid of paragraph, wherein the hepatic-derived cells maintain the expression of P-glycoprotein or wherein the hepatic organoid is genetically modified to decrease expression of P-gp.
7. The hepatic organoid of paragraph 1, wherein the genetic modification comprises a knock-in mutation of an ABCB1-1Δ mutation.
8. The hepatic organoid of paragraph 7, wherein the epithelial-derived cells are diseased; optionally wherein the disease is cancer, cancer cell, or cancer tissue.
9. A culture media for differentiating hepatic stem cells into hepatic organoids, comprising a complete media; and a growth factor, wherein the growth factor differentiates the hepatic stem cell into the hepatic organoid.
10. The culture media of paragraph 9, wherein the growth factor is epidermal growth factor, Noggin, DAPT, dexamethasone, fibroblast growth factor 10 (FGF10), fibroblast growth factor 19 (FGF19), bone morphogenic protein 7 (BMP7), hepatocyte growth factor (HGF), a transforming growth factor beta receptor I inhibitor, a mitogen activated protein kinase 14 inhibitor, and/or combinations thereof.
11. The culture media of paragraph 9, further comprising a rho kinase inhibitor and/or a glycogen synthase kinase 3 inhibitor.
12. The culture media of paragraph 11, wherein the rho kinase inhibitor is Y27632, Y39983, Wf-536, SLx-2119, Azabenzimidazole-aminofurazans, aminopyrimidine, DE-104, olefins, isoquinolines, indazoles, pyridinealkene derivatives, H-1152P, ROKa inhibitors, XD-4000, HMN-1152, 4-(1-aminoalkyl)-N-(4-pyridyl)cyclohexane-carboxamides, Rhostatin, BA-210, BA-207, BA-215, BA-285, BA-1037, Ki-23095, VAS-012, fasudil and/or combinations thereof.
13. The culture media of paragraph 12, wherein one or more growth factors are removed from the culture media, optionally wherein the one or more removed growth factors is ROCKi, CHIR99021, Nicotinamide, R-Spondin, and/or combinations thereof.
14. A canine hepatic organoid culture system, comprising: a three-dimensional canine epithelial organoid, comprising a population of differentiated canine epithelial-derived cells which are capable of organ-like functionality; an extracellular matrix; and a culture media for differentiating stem cells into organoids, comprising a complete media; and a growth factor, wherein the growth factor differentiates the stem cell into the organoid.
15. The canine hepatic organoid culture system of paragraph 14, further comprising a rho kinase inhibitor and/or a glycogen synthase kinase 3 inhibitor.
16. A method of culturing canine hepatic organoid, comprising: obtaining a hepatic sample from a canine; and exposing the sample to a differentiation media.
17. The method of paragraph 16, further comprising isolating a stem cell of interest from the hepatic sample; and enriching the sample for stem cells.
18. The method of paragraph 16, further comprising seeding the sample into an extracellular matrix.
19. The method of paragraph 18, wherein between about 20 to about 200 cells are seeded into the extracellular matrix, or similar macro and microenvironments.
20. The method of paragraph 19, wherein the extracellular matrix stabilizes the three-dimensional structure of the organoid.
21. The method of paragraph 16, further comprising initially contacting the sample with a protective media.
22. The method of paragraph 21 wherein the sample is contacted with the protective media for about 1 day to about 4 days.
23. The method of paragraph 21, wherein the protective media comprises a rho kinase inhibitor and/or a glycogen synthase kinase 3 inhibitor.
24. The method of paragraph 23, wherein the rho kinase inhibitor is Y27632, Y39983, Wf-536, SLx-2119, Azabenzimidazole-aminofurazans, DE-104, olefins, isoquinolines, indazoles, pyridinealkene derivatives, H-1152P, ROKa inhibitors, XD-4000, HMN-1152, 4-(1-aminoalkyl)-N-(4-pyridyl)cyclohexane-carboxamides, Rhostatin, BA-210, BA-207, BA-215, BA-285, BA-1037, Ki-23095, VAS-012, fasudil and/or combinations thereof.
25. The method of paragraph 23, wherein the glycogen synthase kinase 3 inhibitor is an aminopyrimidine.
26. The method of paragraph 16, wherein the differentiation media comprises: a complete media; and a growth factor, wherein the growth factor differentiates the stem cell into the organoid.
27. The method of paragraph 26, wherein the growth factor is epidermal growth factor, Noggin, DAPT, dexamethasone, fibroblast growth factor 10 (FGF10), fibroblast growth factor 19 (FGF19), bone morphogenic protein 7 (BMP7), hepatocyte growth factor (HGF), a transforming growth factor beta receptor I inhibitor, a mitogen activated protein kinase 14 inhibitor, and/or combinations thereof.
28. The method of paragraph 16, further comprising genetically engineering the sample, optionally wherein the genetic engineering is performed using at least two gRNA molecules.
29. The method of paragraph 28, wherein the genetic engineering is performed prior to exposing the sample to the differentiation media.
30. The method of paragraph 28, wherein the genetic engineering is a DNA modification.
31. The method of paragraph 30, wherein the genetic engineering is performed by Cas variants, TALEN, meganucleases, or Zinc Fingers.
32. The method of paragraph 28, wherein the genetic engineering is a RNA modification.
33. The method of paragraph 32, wherein the genetic engineering is performed by RNAi, LEAPER, or Cas variants.
34. A media for freezing hepatic organoids, comprising: a differentiation media; dimethyl sulfoxide; and fetal bovine serum.
35. The media for freezing hepatic organoids of paragraph 34, wherein the differentiation media is from about 40% to about 60%.
36. The media for freezing hepatic organoids of paragraph 34, wherein the dimethyl sulfoxide is from about 5% to about 15%.
37. The media for freezing hepatic organoids of paragraph 34, wherein the fetal bovine serum is from about 30% to about 50%.
38. The media for freezing hepatic organoids of paragraph 34, wherein the differentiation media comprises a growth factor.
39. The media for freezing hepatic organoids of paragraph 38, wherein the growth factor is epidermal growth factor, Noggin, DAPT, dexamethasone, a transforming growth factor beta receptor I inhibitor, a mitogen activated protein kinase 14 inhibitor, and/or combinations thereof.
40. A method for freezing a hepatic organoid, comprising: extracting the hepatic organoid from culture; resuspending the hepatic organoid in a freezing media, the freezing media comprising: a differentiation media; dimethyl sulfoxide; and fetal bovine serum.
41. The method of paragraph 40, wherein the differentiation media is from about 40% to about 60% v/v.
42. The method of paragraph 40, wherein the dimethyl sulfoxide is from about 5% to about 15% v/v.
43. The method of paragraph 40, wherein the fetal bovine serum is from about 30% to about 50% v/v.
44. The method of paragraph 40, wherein the differentiation media comprises a growth factor.
45. The method of paragraph 444, wherein the growth factor is epidermal growth factor, Noggin, R-spondin-1, DAPT, dexamethasone, a transforming growth factor beta receptor I inhibitor, a mitogen activated protein kinase 14 inhibitor, and/or combinations thereof.
46. The method of paragraph 40, further comprising freezing the hepatic organoid below about -76° C. in a freezer for up to 7 days.
47. The method of paragraph 40, further comprising freezing the hepatic organoid in liquid nitrogen.
48. A method of recovering a hepatic organoid from being frozen, comprising: thawing the frozen hepatic organoid in freezing media; transferring the thawed hepatic organoid in freezing media to a complete media drop wise; pelleting the complete media containing the hepatic organoid by centrifugation; aspirating both the freezing media and complete media from the pellet; and resuspending the pellet.
49. The method of paragraph 48, wherein the pellet is resuspended in an extracellular matrix.
50. The method of paragraph 49, further comprising: transferring the resuspended pellet to a cell culture plate; allowing sufficient time for extracellular matrix to settle; and adding protecting media to the gelled extracellular matrix containing the epithelial organoids.
51. The method of paragraph 50, wherein the protecting media comprises a rho kinase inhibitor and/or a glycogen synthase kinase 3 inhibitor.
52. The culture media of paragraph 51, wherein the rho kinase inhibitor is Y27632, Y39983, Wf-536, SLx-2119, Azabenzimidazole-aminofurazans, DE-104, olefins, isoquinolines, indazoles, pyridinealkene derivatives, H-1152P, ROKa inhibitors, XD-4000, HMN-1152, 4-(1-aminoalkyl)-N-(4-pyridyl)cyclohexane-carboxamides, Rhostatin, BA-210, BA-207, BA-215, BA-285, BA-1037, Ki-23095, VAS-012, fasudil and/or combinations thereof.
53. The culture media of paragraph 51, wherein the glycogen synthase kinase 3 inhibitor is an aminopyrimidine.
54. A method for detecting the differences between a healthy hepatic organoid and a diseased hepatic organoid, comprising: obtaining a healthy sample; obtaining a diseased sample; culturing the samples in a differentiation media to form a healthy organoid and a diseased organoid; detecting the expression level of a RNA, protein, and/or the concentration of a metabolite in the healthy organoid and the diseased organoid.
55. The method of paragraph 54, wherein the healthy sample and the diseased sample are taken from the same subject.
56. The method of paragraph 54, wherein the healthy sample and the diseased sample are taken from different subjects.
57. The method of paragraph 54, wherein the detecting is of RNA expression.
58. The method of paragraph 54, wherein the detecting is of protein expression.
59. The method of paragraph 54, wherein the detecting is of metabolite expression.
60. The method of paragraph 54, further comprising administering to the samples an environmental and/or dietary trigger.
61. The method of paragraph 54, wherein the differentiation media comprises a growth factor.
62. The method of paragraph 61, wherein the growth factor is epidermal growth factor, Noggin, DAPT, dexamethasone, a transforming growth factor beta receptor I inhibitor, a mitogen activated protein kinase 14 inhibitor, and/or combinations thereof.
63. A method for detecting the differences between a healthy hepatic organoid and a genetically modified hepatic organoid, comprising: obtaining at least two hepatic samples; genetically modifying one or more of the hepatic samples; culturing the samples in a differentiation media; detecting the expression level of a RNA, protein, epigenetics, and/or the concentration of a metabolite in the healthy sample and the diseased sample.
64. The method of paragraph 63, wherein the healthy sample and the sample to be genetically modified are obtained from the same subject.
65. The method of paragraph 63, wherein the healthy sample and the sample to be genetically modified are obtained from different subjects.
66. The method of paragraph 63, wherein the detecting is of RNA expression.
67. The method of paragraph 63, wherein the detecting is of protein expression.
68. The method of paragraph 63, wherein the detecting is of metabolite expression.
69. The method of paragraph 63, further comprising administering to the samples an environmental and/or dietary trigger.
70. The method of paragraph 68, wherein the differentiation media comprises a growth factor.
71. The method of paragraph 70, wherein the growth factor is epidermal growth factor, Noggin, DAPT, dexamethasone, a transforming growth factor beta receptor I inhibitor, a mitogen activated protein kinase 14 inhibitor, and/or combinations thereof.
72. The method of paragraph 63, wherein the genetic engineering is a DNA modification.
73. The method of paragraph 72, wherein the genetic engineering is performed by Cas variants, TALEN, meganucleases, or Zinc Fingers.
74. The method of paragraph 63, wherein the genetic engineering is a RNA modification.
75. The method of paragraph 74, wherein the genetic engineering is performed by RNAi, LEAPER, or Cas variants.
76. A method of screening drug absorption in the canine liver, comprising: obtaining a sample of canine liver; optionally, genetically modifying the sample to alter the expression of P-glycoprotein (P-gp); culturing the sample in differentiation media to form an organoid; administering a drug; allowing sufficient time for absorption into the organoid lumen; and detecting the concentration of the drug in the lumen and/or intracellular space of the organoid.
77. The method of paragraph 76, further comprising administering a P-glycoprotein interacting compound to the organoid.
78. The method of paragraph 77, wherein the P-glycoprotein interacting compound is an inhibitor.
79. The method of paragraph 77, wherein the P-glycoprotein interacting compound is a substrate.
80. The method of paragraph 77, wherein the P-glycoprotein interacting compound is an inducer.
81. The method of paragraph 76, wherein two or more samples are obtained at different time points from the same subject.
82. The method of any one of paragraph 76, wherein detecting is measured by fluorescence, a binding assay, through high performance liquid chromatography, and/or staining.
83. The method of paragraph 76, further comprising genetically modifying P-glycoprotein.
84. The method of paragraph 76, wherein the genetic-modification alters the expression level of P-glycoprotein, optionally wherein the genetic modification decreases expression of P-glycoprotein.
85. The method of paragraph 76, wherein the genetic-modification alters the binding kinetics of P-glycoprotein.
86. The method of paragraph 81, wherein the differentiation media comprises a growth factor. Growth factors may include EGF, FGF, NGF, PDGF, VEGF, IGF, GMCSF, GCSF, TGF, Erythropoietin, TPO, BMP, HGF, GDF, Neurotrophins, MSF, SGF, GDF, and other growth factors known in the art.
87. The method of paragraph 91, wherein the growth factor is epidermal growth factor, Noggin, DAPT, dexamethasone, fibroblast growth factor 10 (FGF10), fibroblast growth factor 19 (FGF19), bone morphogenic protein 7 (BMP7), hepatocyte growth factor (HGF), a transforming growth factor beta receptor I inhibitor, a mitogen activated protein kinase 14 inhibitor, and/or combinations thereof.
88. The method of paragraph 76, wherein the genetic modification comprises a knock-in mutation of an ABCB1-1Δ mutation.
89. The method of paragraph 76, wherein the genetic modification is conferred using at least two gRNA molecules.
90. A two-dimension membrane organoid, comprising of: a population of differentiated canine hepatic-derived cells which are capable of organ-like functionality; and a membrane.
91. The two-dimension membrane organoid of paragraph 90, wherein the membrane is permeable.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims. The disclosures of each and every patent, patent application, and publication cited herein are hereby incorporated herein by reference in their entirety.

The following examples are not intended to limit the scope of the claims.

### Example 1

Developing systems for studying drug hepatic transport and metabolism is critical for predicting bioavailability of therapeutic drugs in medicine. Specifically, conventional systems such as 2D epithelial cultures utilizing cancer-derived cell lines (e.g., Caco-2, T84, and HT29), or spontaneously immortalized epithelial cells do not faithfully reproduce the structure and function of hepatocytes or cholangiocytes. Since such systems do not express the same transporters, such as canine P-gp, there is the risk for incorrect conclusions associated with substrate specificity, drug-drug interactions, or enzyme kinetics. Specifically, Caco-2 cells are a human colon adenocarcinoma cell line and are not derived from canine tissues. Therefore, any data generated using Caco-2 will have uncertain relevance to models of canine oral drug absorption and metabolism.

Because in vitro 3D cell culture systems provide a more realistic translation to in vivo conditions than do most 2D culture systems, 3D hepatic organoids will better harness the complexity of the in vivo biology. Accordingly, this would provide an opportunity to conduct in vitro mechanistic studies for evaluating drug absorption. However, the molecular characteristics of the organoids has not been assessed, particularly for P-gp. Therefore, it is essential to assess the localization, expression, and function of P-gp in 3D models, such as canine ileal organoids.

### Material and Methods

### Hepatic Tissue and Stem Cell Isolation for Culture of Hepatic Organoids

Hepatic stem cells were obtained and histological evaluation from healthy research colony dogs. Briefly, samples were cut into small pieces and hepatic stem cells were released by incubating the samples with complete chelating solution and EDTA (30 mM) for 60 min at 4° C. After release, the stem cell-containing pellet was suspended and seeded in 30 μL per well of MATRIGEL® (CORNING® MATRIGEL® Growth Factor Reduced (GFR) Basement Membrane Matrix) and 500 μL per well of complete medium with hepatic stem cell (HSC) growth factors (CMGF+) supplemented with 10 μM rho-associated kinase inhibitor (ROCKi) Y-27632 (Stem-Gent) and 2.5 μM glycogen synthase kinase 313 (GSK3(3) inhibitor CHIR99021 (StemGent) before the plate was incubated at 37° C. The culture medium was changed to CMGF+ without any supplement after 2 days of crypt isolation, while passage and expansion of organoids were performed with TrypLE Express treatment at 37° C. for 10 min. Cells were cultured for up to 11 days.

Once stable organoid cultures were established, representative hepatic organoids were fixed with 10% formalin and stored in 70% ethanol for IHC staining and RNAscope® 2.5 detection. All the formalin fixed samples were paraffin embedded and cut into 3-μm sections for placement onto glass slides.

### Immunohistochemistry (IHC)

Immunohistochemistry (IHC) assays were performed based on a commercially available protocol at the Iowa State University Veterinary Diagnostic Laboratory (Discovery Ultra, Ventana Medical Systems, Inc.). Briefly, paraffin-embedded sections were first deparaffinized and rehydrated, followed by antigen retrieval and blocking steps. The sections were incubated with primary antibodies (LGRS, KRT7, CYP3A12, and CYP3A4), followed by Diaminobenzidine (DAB) staining reagents and subsequently treated with hematoxylin counterstaining. Image acquisition was performed using the Olympus CellSens Standard Ver.1.18 (Tokyo, Japan), while semi-quantitative image analysis of DAB detection was performed using the ImageJ v1.52q15. The quantified DAB staining was controlled by the hematoxylin counterstaining to control for the number variation of the cell number within an image.

### RNAscope

Paraffin embedded samples were also assayed for the cell differentiation markers of KRT7, LGRS, AQP1, FOXA1, and CYP3A12 using RNAscope® 2.5 following the manufacturers protocols.

### Example 2

A variety of organoid media and Formalin-Acetic Acid-Alcohol (FAA) solutions are disclosed herein. Due to obtaining the various cell lines in the Example 1, specifically cholangiocytes and hepatocytes, and maintaining stem cells within the cultures, it is possible to optimize the CMGF+ media. For example, one may alter the concentrations and/or timing of administering the growth factors to enrich for either hepatocytes or cholangiocytes, as described further in Example 3.

The complete composition of CMGF+, CMGF+ R/G, and FAA are summarized in Table 2 below.

TABLE 2
Organoid Media

| | Final concentration | |
|---|---|---|
| Organoid media composition | | |
| Advanced DMEM/F12 | NA | |
| FBS | 8% | |
| Glutamax | 2 | mM |
| HEPES | 10 | mM |
| Primocin | 100 | μg/ml |
| B27 supplement | 1× | |
| N2 supplement | 1× | |
| N-Acetyl-L-cysteine | 1 | mM |
| Murine EGF | 50 | ng/ml |
| Murine Noggin | 100 | ng/ml |
| Human R-Spondin-1 | 500 | ng/ml |
| Murine Wnt-3a | 100 | ng/ml |
| [Leu¹⁵]-Gastrin I human | 10 | nM |
| Nicotinamide | 10 | mM |
| A-83-01 | 500 | nM |
| SB202190 (P38 inhibitor) | 50 | μM |
| TMS (trimethoprim sulfate) | 10 | μg/ml |
| Additional components | | |
| ROCK inhibitor (Y-27632) | 10 | μM |
| Stemolecule CHIR99021 (GSK3β) | 2.5 | μM |

| FAA composition | V/V percent |
|---|---|
| Ethanol (100%) | 50% |
| Acetic Acid, Glacial | 5% |
| Formaldehyde (37%) | 10% |
| Distilled water | 35% |

Further, preferred media for organoid monolayer cultures is summarized in Table 3 below. In embodiments, CMGF+ R/G in Transwells is changed in the apical and basolateral chambers three times per week (e.g., Monday, Wednesday and Friday of each week). The longer cultivation period over the weekend demands an increased volume of media in both apical and basolateral chambers given on Friday afternoon with a media change on Monday morning.

TABLE 3

| | | |
|---|---|---|
| Name of Material/Equipment | Company | Catalog Number |

Organoid media

| | | |
|---|---|---|
| ROCK inhibitor (Y-27632) | EMD Millipore | SCM 075 |
| | Corp. | |
| [Leu¹⁵]-Gastrin I human | Sigma | G9145-.5MG |
| A-83-01 | PeproTech | 9094360 |
| Advanced DMEM/F12 | Gibco | 12634-010 |
| B27 supplement | Gibco | 17504-044 |
| FBS | Corning | 35-010-CV |
| Glutamax | Gibco | 35050-061 |
| HEPES | VWR Life Science | J848-500ML |
| Human R-Spondin-1 | PeproTech | 120-38-500UG |
| Murine EGF | PeproTech | 315-09-1MG |
| Murine Noggin | PeproTech | 250-38-250UG |
| Murine Wnt-3a | PeproTech | 315-20-10UG |
| N2 supplement | Gibco | 17502-048 |
| N-Acetyl-L-cysteine | Sigma | A9165-25G |
| Nicotinamide | Sigma | N0636-100G |
| Primocin | InvivoGen | ant-pm-1 |
| SB202190 (P38 inhibitor) | Sigma | S7067-25MG |
| Stemolecule CHIR99021 | Reprocell | 04-0004-base |
| (GSK3β) | | |
| TMS (trimethoprim sulfate) | Sigma | T7883-5G |

Reagents

| | | |
|---|---|---|
| Acetic Acid, Glacial | Fisher Chemical | A38-500 |
| Formaldehyde (37%) | Fisher Chemical | F79P-4 |
| Matrigel Matrix For Organoid | Corning | 356255 |
| Culture | | |
| PBS, 1× (Phosphate-Buffered | Corning | 21-040-CM |
| Saline) | | |
| Paraformaldehyde, 97% | Alfa Aesar | A11313 |
| Glutaraldehyde solution | Sigma | G5882 |
| TrypLE Express | Gibco | 12604-021 |
| Cell Recovery Solution | Corning | 354253 |
| Collagen I, Rat Tail 3 mg/mL | Gibco | A10483-01 |
| FITC-CM-Dextran | Millipore Sigma | 68059-1G |
| HBSS (1×) | Gibco | 14025-076 |
| pha-D(+)-Glucose, 99+%, | Acros Organics | 170080010 |
| anhydro [symbol] | | |

Materials and Equipment

| | | |
|---|---|---|
| 9″ Pasteur Pipets | Fisherbrand | 13-678-6B |
| Millicell ERS-2 Voltohmmeter | Millipore Sigma | MERS00002 |
| Millicell ERS (Probes) | Millipore Sigma | MERSSTX01 |
| Corning Transwell 6.5 mm | | |
| Polyester Membrane Inserts Pre- | Corning | 3470 |
| Loaded in 24-Well Culture | | |
| Plates, Pore Size: 0.4 μm, Sterile | | |

[symbol]

indicates data missing or illegible when filed

### Example 3

Previous descriptions of hepatic organoid cultures described these cultures to consist of cholangiocytes (characterized by cytokeratin 19/KRT-19 expression) in their initial growth stages. These 3D organoids typically lack a larger population of differentiated mature hepatocytes, which would represent metabolically active liver cells, the main cell type responsible for xenobiotic detoxification. Prior protocols resulted in cells that did not fully exhibit mature hepatocyte functions, such as similar albumin production, low-density lipoprotein (LDL) uptake, and glycogen storage as would be expected in vivo. In order to more faithfully model the in vivo ratio of mature cholangiocyte/hepatocytes found in vivo in the liver organoids, it is therefore crucially important to explore different media compositions to drive ASC derived from liver tissue towards enhanced mature hepatocyte differentiation.

### Methods

### Differentiation Component Selection

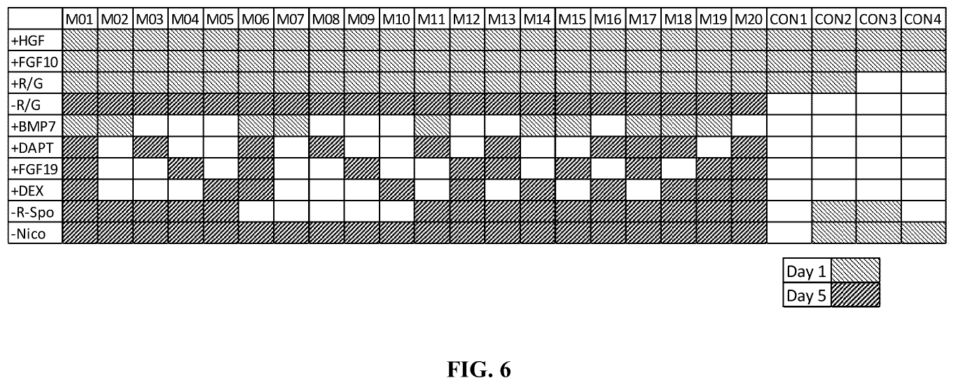

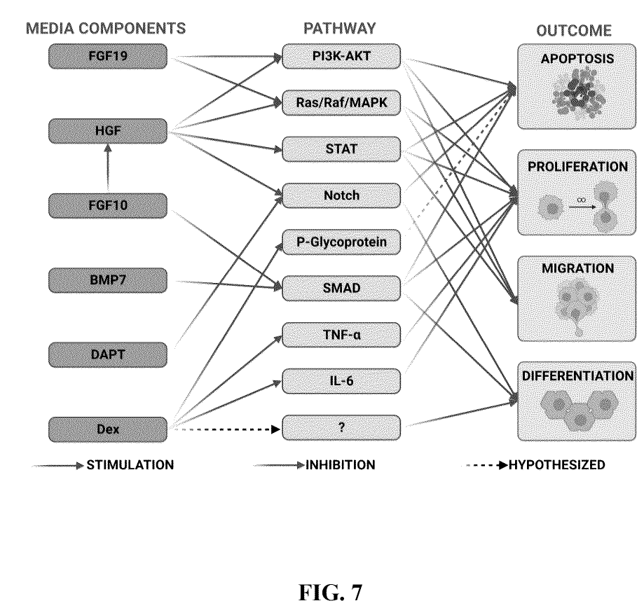

The canine organoid media of Example 2 was supplemented by adding human recombinant hepatocyte growth factor (HGF) and fibroblast growth factor 10 (FGF10). Five additional media constituents were also added: bone morphogenetic protein 7 (BMP7), fibroblast growth factor 19 (FGF19), dexamethasone (Dex), gamma-secretase inhibitor IX (DAPT), and activin-like kinase 5 inhibitor (A8301). Eight media components were removed: Wnt, Rock inhibitor (ROCKi), Noggin, Nicotinamide (Nico), R-Spondin-1 (R-spo), forskolin, HGF, and FGF10 from the organoid expansion media to achieve optimal differentiation conditions. In order to standardize all experimental conditions, the addition of components was always executed on Day 5 after starting the differentiation of the organoids. BMP7, DAPT, Dex, and FGF19 were added and combined in 20 unique differentiation media compositions for comparison. (FIG. 6). The proposed method of action of the added differentiation components is visualized in FIG. 7. Wnt withdrawal was omitted from these experiments because cultures deteriorated rapidly whenever Wnt was removed.

### Cell Count and Albumin Measurement

Hepatic organoid cell culture lines that had previously been derived from a healthy adult dog were thawed, passaged, and seeded in a 24-well plate at a density of ˜100,000 cells/well. Culture conditions for the growth and maintenance of canine organoids were used as previously described. Experiments were performed in triplicates. Twenty different media compositions were tested, with four organoid media (expansion media with ROCKi/CHIR99021 (GSK3 inhibitor)=R/G; media with R/G w/o R-spo and Nico; media w/o R-spo and Nico; media w/o Nico) serving as positive controls. The hepatocyte differentiation encompassed two phases. The first phase lasted for the initial four days of culture and differentiation, followed by the second phase of culture (change of media composition) spanning from day 5 to day 14. To determine the viability of the organoid culture in each media and to avoid dissociation of the organoid structures, organoids were initially grown without cleaning or passaging, while culture supernatants were harvested on Day 11 and Day 14 after the start of the experiment.

On Day 14, cells were passaged, and their quantity and viability were evaluated for every well individually and in experimental duplicates using an automated cell counter (Countess II FL, Thermo Fisher) and trypan blue staining to assess cell viability, following a protocol published in 2015. Canine albumin concentrations were measured in the culture supernatant using a colorimetric ELISA immunoassay in triplicates (ab277078, Abcam; ELC-Albumin RayBiotech). Albumin production in the culture supernatant was measured following the manufacturer's instructions. Samples were analyzed on a microplate reader (BioRad iMark), and data were plotted using the four-parameter logistic curve method. At this stage, the media yielding the highest concentrations of albumin in the supernatant (>50 ng/mL) were identified.

In subsequent experiments, these six media were used for the culture of organoid cell lines derived from two healthy adult dogs (1M+1F) in 12 replicates. Organoids were first defrosted and expanded in expansion medium as previously described. They were then seeded at a lower density of ˜50,000 cells/well to decrease excessive apoptosis due to overcrowding. Organoids were finally cleaned during differentiation, as previously described. Differentiated organoids were harvested for downstream analysis to characterize the phenotypic and functional differentiation status of the cells, including histopathology, morphological examination by bright field microscopy, transmission electron microscopy, RNAScope for selected markers of differentiation, immunofluorescence as well as protein analysis in the supernatant using matrix-assisted laser desorption/ionization-time of flight (MALDI-TOF) mass spectrometry.

### Label-Free Relative Quantitative Proteomics

The supernatant samples of culture M01 and the control were harvested and analyzed at the Iowa State University Office of Biotechnology Protein Facility. A label-free relative quantitative proteomics mass spectrometry method was performed to quantify the protein abundance ratio in the supernatant samples. The sequence of bovine serum albumin protein (BSA), which was abundantly present in the media, was searched using Mascot against Sprot-mammalia and with Sequest HT against Sprot-a1127. The samples were searched with Mascot against Sprot-Canis lupus familiaris and Sequence HT against the PRTC sequences. The protein abundance ratio was used to identify the most differentially expressed proteins between organoids cultured in the differentiation media (BMP7, DAPT, FGF19, and Dex) and the organoids cultured in the control media.

### Single-Molecule RNA In Situ Hybridization

RNAscope was utilized to perform RNA in situ hybridization experiments. In short, the following manufacturer-designed paired double-Z oligonucleotide probes against the canine target RNA were used: Cl-LRG5 (cat no. 405651, XM_846738.2, nt 517-1506), Cl-KRT7 (cat no. 838971, XM_005636798.2, nt 418-1640), Cl-CYP3A12 (cat no. 577821, NM 001003340.1, nt 2-1823) Cl-CYP2B6/CYP2B11 (cat no. 577831, NM_001006652.1, nt 121-1207) and Cl-Polr2a (cat no. 310981, XM_852751.3, nt 1846-2924) as a positive control. The RNAscope 2.5 HD Reagent Kit-RED (cat no. 322350, Advanced Cell Diagnostics, Newark, CA) was used according to the manufacturer's instructions. FFPE sections were prepared based on the manufacturer's recommendations. Brightfield images (N>10) were acquired on an Olympus BX43 microscope using a 40× objective. Fiji software was used for signal/cell area quantitative measurements following the manufacturer's technical note.

### Histological Staining and Description

For paraffin-embedding of the organoids, culture media was removed from the wells, and an FAA solution (Formalin-Acetic Acid-Alcohol) was added to the samples still embedded in the extracellular membrane matrix (ECM; Matrigel for Organoids, Phenol Red-free Matrigel; Corning). After 24 hours, FAA was removed, and 70% ethanol was added before samples were sent for embedding in traditional metal base molds and staining at the Veterinary Diagnostic Laboratory of Iowa State University. Staining included Hematoxylin & Eosin (H&E), Periodic acid—Schiff (PAS), PicroSirius Red, Masson's Trichrome, periodic acid silver methenamine (PASM), and Iron stain (Prussian Blue stain). H&E-stained slides were provided to a histopathologist who was blinded to the identity of the samples to compare the percentage of canine organoid cells morphologically resembling cholangiocytes or hepatocytes, respectively, for each of the experimental media used. The slides were the deparaffinized/hydrated. Specific staining protocols can be found in Supplementary Material.

### Transmission Electron Microscopy

Samples were placed in 1% paraformaldehyde, 3% glutaraldehyde in 0.1M sodium cacodylate buffer, at pH 7.2 and fixed for 48 hours at 4° C. Samples were washed in cacodylate buffer 3 times/10 minutes each, and post-fixed with 1% osmium tetroxide in 0.1M sodium cacodylate buffer for 1 hour at room temp. Samples were washed with deionized water 3 times for 15 minutes each, and en-bloc stained using 2% uranyl acetate in distilled water for 1 hour. Samples were washed in distilled water for 10 minutes and dehydrated through a graded ethanol series (25, 50, 70, 85, 95, 100%) for 1 hour per step. Samples were further dehydrated with 3 changes of pure acetone, 15 minutes each, and infiltrated with EmBed 812 formula (hard) for EPON epoxy resin (Electron Microscopy Sciences, Hatfield PA) with graded ratios of resin to acetone until fully infiltrated with pure epoxy resin (3:1, 1:1, 1:3, pure) for 6-12 hours per step. Tissues were placed into beem capsules and were polymerized at 70° C. for 48hours. Thick sections (1.5 um) were cut using a Leica UC6 μltramicrotome (Leica Microsystems, Buffalo Grove, IL) and stained with EMS Epoxy stain (a blend of toluidine blue-Oand basic fucsin). Thin sections were cut at 50 nm and collected onto single slot carbon film grids. TEM images were collected using a 200 kV JEOL JSM 2100 scanning transmission electron microscope (Japan Electron Optics Laboratories, USA, Peabody, MA) with a GATAN One View 4K camera (Gatan inc., Pleasanton, CA).

### Immunofluorescence

Slide deparaffinization was achieved by washing in xylene twice for ten minutes, followed by 100% ethanol wash twice for one minute. Washes were performed with aggressive agitation. After deparaffinization was complete, the slides were allowed to air dry for five minutes and Heat-Induced Epitope Retrieval (HIER) was started with a Citrate buffer (pH 6) for two hours at 75° C. using a HybEZ II Oven. This step was performed to remove any remaining bonds from the paraffin embedding process. After the HIER was complete, slides were allowed to cool to room temperature for 15 minutes and rinsed twice in phosphate-buffered saline (PBS) for two minutes each. One last wash was performed in PBS for ten minutes and then the slides were permeabilized via incubation in 0.25% Triton in PBS for twenty minutes. Slides were rinsed in PBS three times to get any remaining Triton off them and then blocked for one hour at room temperature in Casein in PBS. Once the blocking step was complete slides were allowed to sit overnight in p-glycoprotein at 1:50 dilution in 4° C. Once incubation was complete, samples were rinsed three times in PBS and incubated in donkey anti-rabbit secondary at 1:1000 for one hour. For nuclear staining, 4′,6-diamidino-2-phenylindole (DAPI) at 1:500 was added to the samples for 20 minutes at room temperature. Slides were washed again in PBS for ten minutes each three times. A last rinse was performed with distilled water, and then slides were dried and mounted with Fluoroshield (Sigma, F6182-20mL). All images were taken on a Stellaris Confocal Microscope.

### Statistical Analysis

Raw data of the cell count was freed from outliers using the ROUT method (Q=10%) and results were presented with standard deviation (SD). The adjusted values of albumin count to cell number are presented with SD. At least 10 representative RNAscope images of each marker were taken, and signal area/cell signal area was counted for every individual picture. Outliers were identified using ROUT method (Q=10%) and D′Agostino & Pearson test was assessed normality. Statistical significance (P<0.05) was assessed using ordinary one-way ANOVA. SD is represented as a±value.

### Results

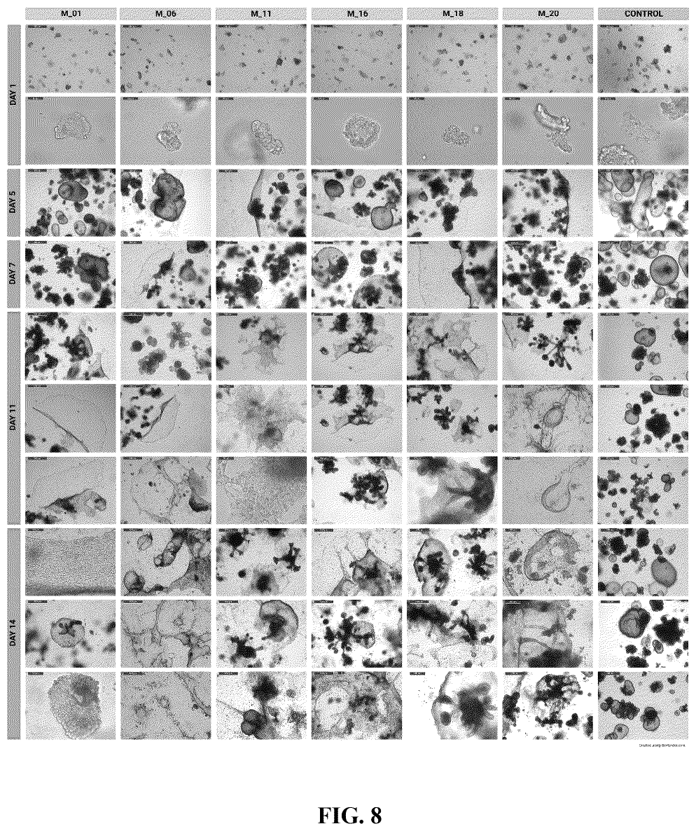

Organoid Morphology: Distinct differences in organoid morphology were identified during hepatocyte differentiation using different media compositions; a summary of morphologies can be seen in FIG. 8. Spheroids formed and rapidly expanded in size on the second day of the third passage. By Day 5 of culture, all of the experimental media used resulted in an altered morphology of the organoids compared to the control media, including monolayer-like growth around the extracellular basement membrane drop (a phenomenon previously described as epithelial-to-mesenchymal transition). While the control group treated with organoid expansion media alone resulted in the expansion of mostly spheroids and only a few more differentiated, budding structures, the organoid cell lines cultured in the experimental media differentiated mainly into budding structures with only a few spheroids. Apoptotic changes in the organoids were present in all experimental samples by Day 7 of culture. This change was predominantly seen in the center of the extracellular basement membrane drop, while organoids on the outside of the extracellular membrane matrix seemed more proliferative and less apoptotic. Structures that partially transdifferentiated into the monolayer plaques showed no signs of apoptosis. By day 7, experimental and control samples contained a high density of medium-to very-large organoids based on scoring previously described in a standardized protocol.

Interestingly, the organoids in M16 showed entirely different morphologies, with groups of traditional budding organoids forming larger common spheroid megastructures. These organoids transitioned into fibroblast/mesenchymal-resembling cells and formed a monolayer expanding into a halo around the basement membrane drop by Day 11. Organoid samples of the control group (CMGF+ R/G) did not change in morphology but began to undergo apoptosis by Day 11, likely due to the lack of passaging and overcrowding, which was expected due to the sequence of the experimental layout. All organoids in experimental samples suffered complete apoptosis of cells in the central section of the Matrigel drop but created interconnected structures closer to the drop border, changing into cellular monolayers escaping the ECM border (FIG. 8, Day 14). M01 resulted in the highest organoid proliferation with the least amount of apoptosis out of all experimental samples, as assessed by cell count (265,000±32,527 cells/well) and observation of the cells via brightfield microscopy. By Day 14, M01-cultured organoids created massive plaques of well-recognized cells and distinct organoids consisting of cell clusters without a lumen (FIG. 8, Day 14). Cells cultured in M06 expanded into a monolayer-like structure with a set of interconnected tubular structures with chambers. M11, M16, M18, and M20 created distinct types of organoids characterized by tubular growth out of the organoid center and expansion from the outer edge of the organoid structures (FIG. 8, Day 14). M20 seemed to express the most elaborate structures of interconnected tubules and chambers out of all media tested (FIG. 8, Day 14). Apoptosis occurred on Day 14 in all samples, including control, due to the lack of organoid passaging and regular cleaning.

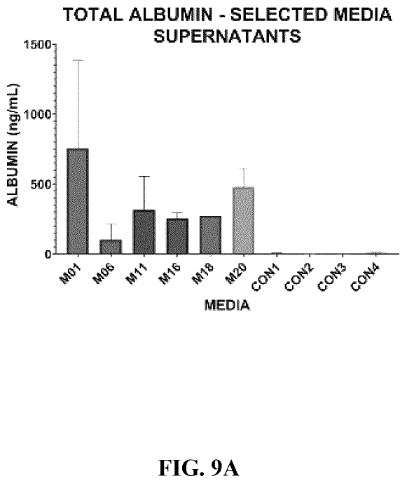

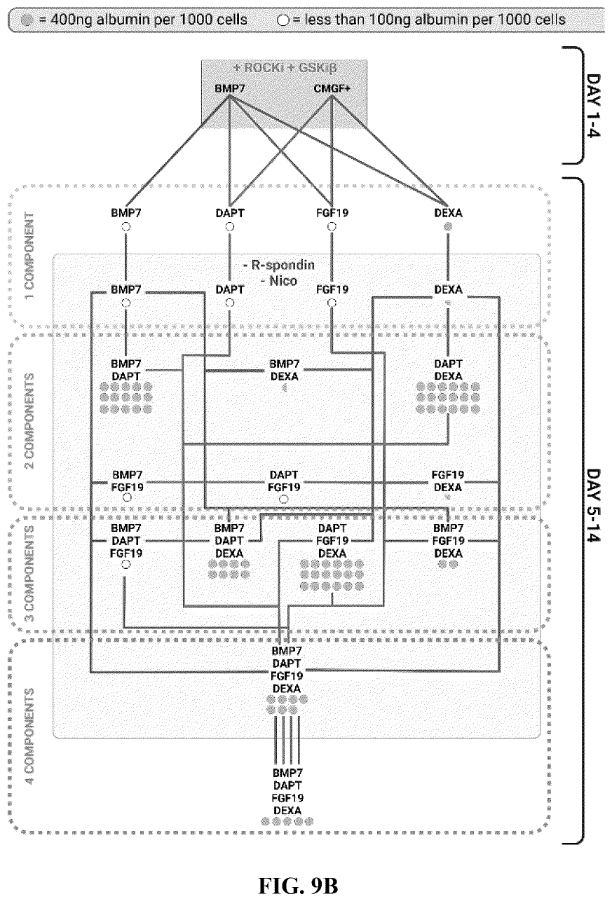

Albumin Production: The highest average albumin yield in the 20 different media experiments was achieved in M01 (757±625 ng/mL), followed by M20 (480±135 ng/mL), Mll (316±243 ng/mL), M18 (273 ng/mL), M16 (254±40 ng/mL), and M06 (104±110 ng/mL). M18 was not measured in triplicates due to loss of the organoid cells during sample processing. Albumin concentrations of 100 ng/mL served as a cutoff for inclusion in the final experiment. The mean concentrations of albumin in the supernatant of the other two media ranged from 30±54 ng/mL for M19 to 0.5±0.4 ng/mL for M04 media. At the same time, albumin concentrations in the control media ranged from 6.3±7.8 ng/mL to 0.9±0.4 ng/mL. The average albumin concentration for the media that was selected as the final experimental media was 387±351 ng/mL, with the concentration average in the control media being 2.8±3.6 ng/mL, and the average albumin concentration for the excluded media at 6.7±10.3 ng/mL. Results on albumin production are summarized in FIGS. 9A-9B.

Albumin concentrations in the supernatant was normalized against the live cell count per well using the trypan blue exclusion method. M16 had the highest albumin production averaging 7,271±1,137 pg/mL/1,000 cells, followed by M20 (7,215±2,024 pg/mL/1,000 cells), Mll (6,094±4,701 pg/mL/1,000 cells), M18 (3,256 pg/mL/1,000 cells), M01 (2,857±2,358 pg/mL/1,000 cells), and M06 (2,037±2,164 pg/mL/1,000 cells). Control media albumin concentration in the supernatant varied between 13.54±5.5 pg/mL/1,000 cells of CON2 to 399.1±489.2 pg/mL/1,000 cells of CON4. The mean albumin concentrations for the two media excluded in the final experiments ranged from 828.6±14.86 pg/mL/1,000 cells for M19 to 4.7±3.3 for M04. When the mean albumin concentration was normalized against cell counts for the group of “selected media,” the results were 5,030±3,161 pg/mL/1,000 cells, 97±216 pg/mL/1,000 cells for control media, and 133±229 pg/mL/1,000 cells for the two excluded media. For comparison, the differentiation media in Huch's human hepatic organoid media protocol yielded approximately 350 ng ALB/day/10⁶ cells.

RNA Scope: RNA in situ hybridization was performed for canine LGRS, CYP2B11, CYP3A12, and SOX9 and was expressed as signal area/cell area percentage. LGRS mRNA expression in organoids cultured in various differentiation media ranged from 0.003±0.004% to 0.057±0.031% (M11), while the control group averaged an expression of 9.846±10.5%. LGR5 expression by RNAscope in the donor hepatic tissue was 2.856±3.631%. SOX9 expression averaged 2.773±1.478% for M11, while the control averaged 5.07±6.048%. The rest of the differentiation medias ranged from 0.023±0.019% (M16) to 0.742±0.683% (M06) for SOX9. CYP2B11 expression was highest in M01 (2.462±2.757%), and M16 (1.998±1.821%). Other differentiation media expressed CYP2B11 at lower levels ranging from 0.052±0.052 (M11) to 0.302±0.333% (M20). CYP2B11 was not expressed in a control group. Finally, CYP3A12 was expressed highest in M16 (0.322±0.167%) and lowest in the control (0.013±0.011%). The rest of the differentiation media expressed CYP3A12 in a range from 0.137±0.080% (M11) to 0.202±0.082% (M06).

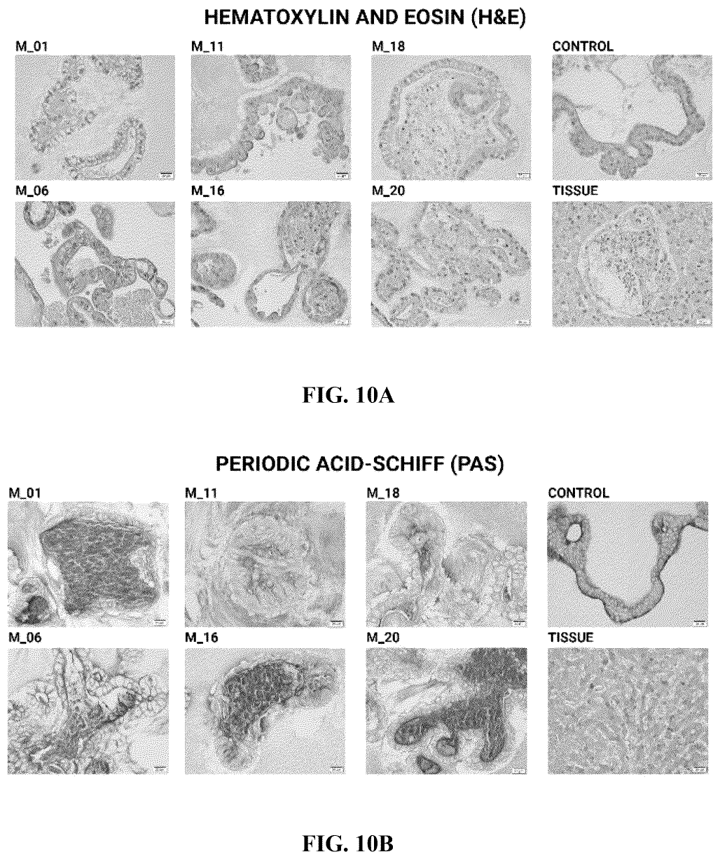

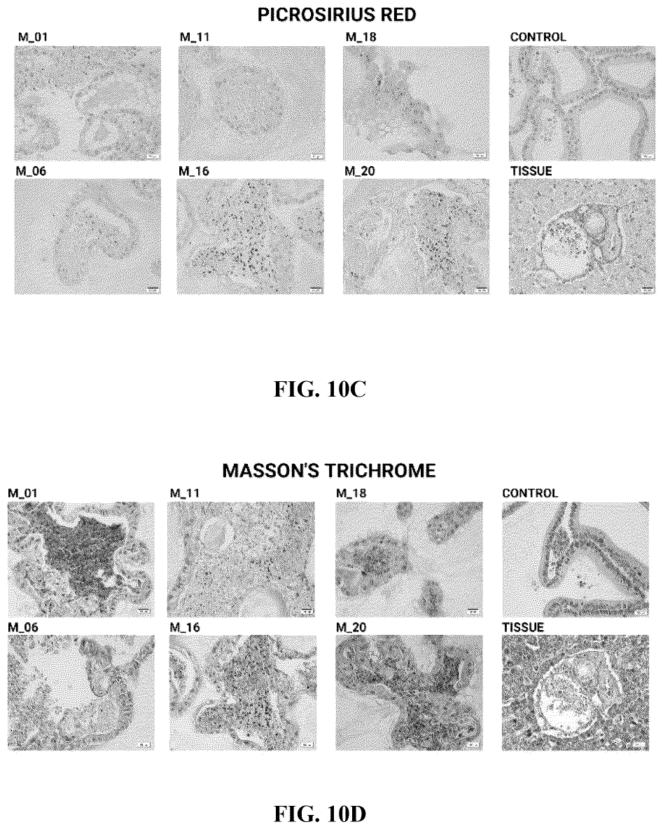

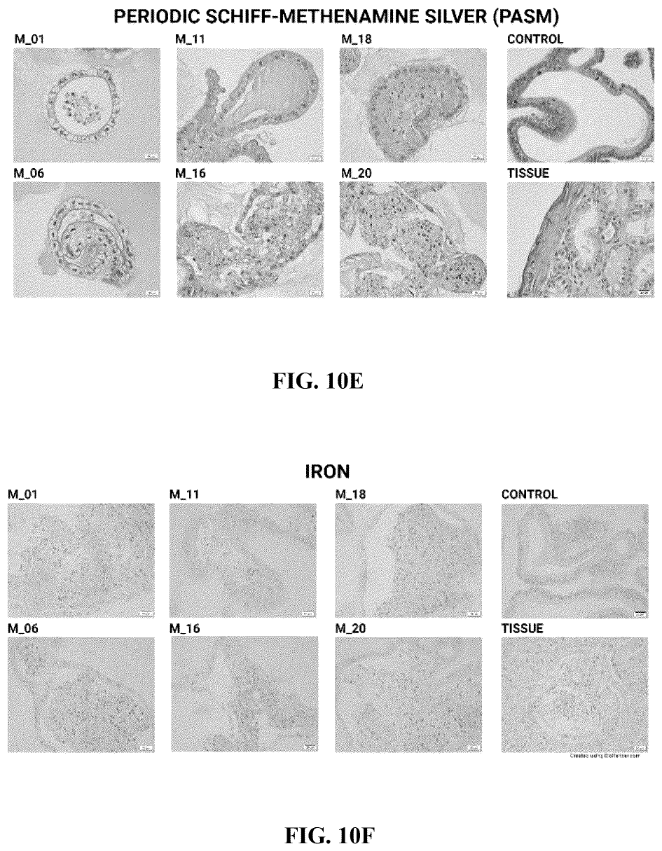

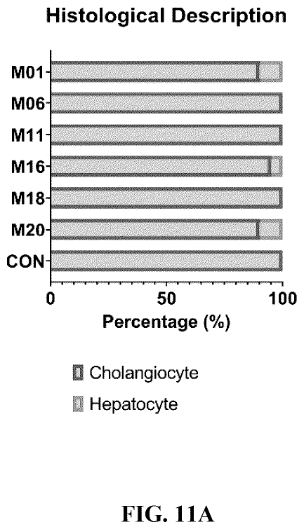

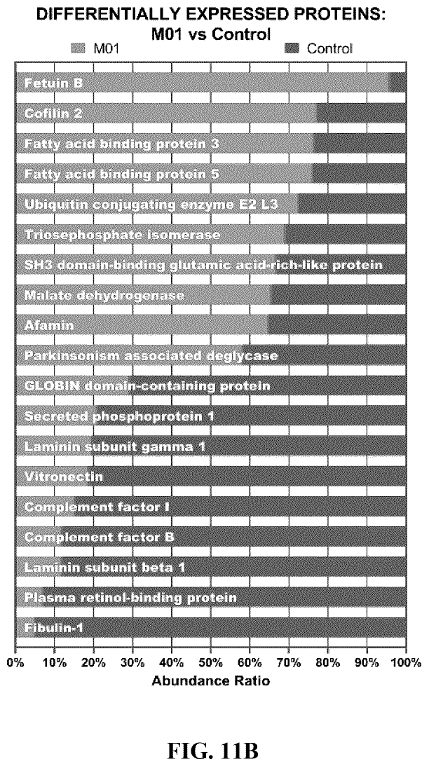

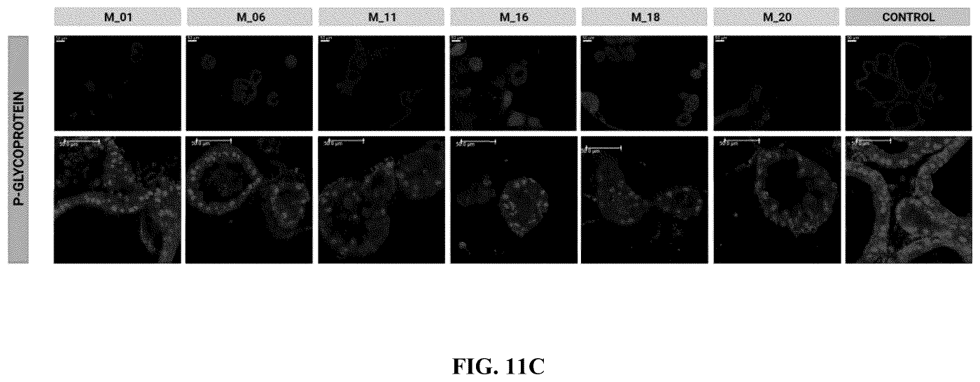

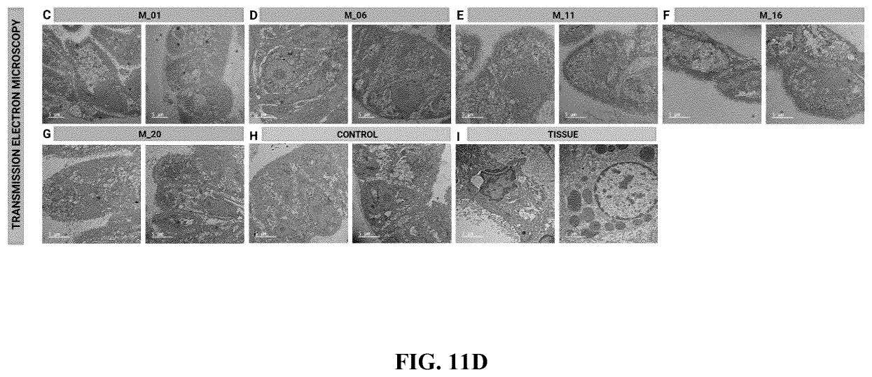

Histologic Staining: Organoids were stained with H&E, PAS, PicroSirius Red Stain, Masson's trichrome, PASM, and Prussian Blue (FIGS. 10A-10F). H&E staining was used to describe general morphology characteristics. More than 90% of cells were identified as cholangiocytes in organoids cultured in all of the experimental media conditions, while the control group was almost entirely composed of cells resembling cholangiocytes. Other samples contained up to 10% of differentiated hepatocytes, including M01 (10%), M16 (5%), and M20 (10%) (FIG. 11A). Hepatocytes were identified as polyhedral cells with acidophilic cytoplasm that contained dotted basophilic regions representing rough endoplasmic reticulum and ribosomes. The center of the cells contained between two and four large and spherical nuclei containing at least two nucleoli. Cholangiocytes were identified as variable in shape from cuboidal to columnar and having less dense eosinophilic cytoplasm, and containing a single central dense basophilic nucleus with a single nucleolus. The apical membrane aspect of these cholangiocyte-like cells was caved in, and in some cases microvilli were observed. Most cells expressed ballooning degeneration and accumulation of lipid droplets.

Masson's trichrome was used to stain for collagen I and showed more hepatocyte background in the tested media than in control media cultured organoids (FIGS. 10A-10F). Collagen is primarily produced by hepatocytes and fat-storing cells. Collagen was detected in cellular monolayers and in debris located inside the organoid structures. Cells cultured in M16 expressed lower number of cells with ballooning degeneration than controls, while cells cultured in M25 expressed high collagen production combined with a high degree of ballooning degeneration. Control group organoids (cultured in M25) did not express any ballooning degeneration of cells. PAS staining to identify basement membrane was not found in any organoids examined but was found in tissue samples, indicating the absence of basement membrane structures in the organoid cultures. Prussian Blue staining was performed to detect excessive iron accumulation and was not found to be present in the organoids or the original tissue, but was detected in the positive control tissue. Picrosirius red was used to stain for collagen I and III nonexclusively red and cellular cytoplasm yellow. Picrosirius red was much more highly positive for collagen staining in differentiation media-treated organoids than in controls. PAS staining was used for detection of glycogen (red to magenta color) and was not identified in any of the slides.

Label-Free Relative Quantitative Proteomics: Proteomics was used to identify and quantify additional proteins in one select media (M01) and was compared to the supernatants of the control cultures. The protein abundance in the supernatant was investigated for M01 and correlated to the control sample (expressed as protein abundance ratio). M01 was chosen to represent a traditionally used component combination. Differentially expressed proteins between the two media were defined as more than 10% difference in abundance of specific proteins between M01 and control media, and included Fetuin B (FETUB), Cofilin 2 (CFL2), Fatty acid binding protein 3 (FABP3), Fatty acid binding protein 5 (FABPS), Ubiquitin-conjugating enzyme E2 L3 ,UBE2L3), Triosephosphate isomerase (TPI), SH3 domain-binding glutamic acid-rich-like protein (SH3BGRL), Malate dehydrogenase (MDH1), and Afamin (AFM). Most differentially expressed proteins in the controls compared to the M01 treated sample consisted of Fibulin-1 (FBLN1), Plasma retinol-binding protein (RBP), Laminin subunit beta 1 (LAMB1), Complement factor B (CFB), Complement factor I (CFI), Vitronectin (VTN), Laminin subunit gamma 1 (LAMC1), Secreted phosphoprotein 1 (SPP1), GLOBIN domain-containing protein, and Parkinsonism associated deglycase (PARK7). These results are summarized in FIG. 11B.

Transmission Electron Microscopy: Transmission electron microscopy images can be appreciated in FIG. 11C. When compared to the donor tissue, the organoids do not exhibit full differentiation into morphologically mature hepatocytes. Numerous cells with secretory granules are observed and organoids maintain their 3D monolayer microarchitecture. While the organoid cells overall sustained their cholangiocyte-like morphology, several markers of some degree of hepatocyte differentiation were also observed, including redundant mitochondria that are normally found in hepatocytes, the presence of secretory granules in the cells which is reminiscent of hepatocytes producing bile, and the formation of bile canaliculi of adjacent hepatocytes.

Immunofluorescence: Samples were successfully assessed for the presence of P-glycoprotein (PGP) protein. Control group expressed higher amount of PGP in comparison to every other experimental group by visual inspection (FIG. 11D).

### Discussion

Hepatic organoids derived from adult stem cells from liver tissues of different species have previously been described to be primarily composed of cholangiocytes/ductal cells, whereas mature hepatocytes are the dominate cell type in liver tissue. This is in contrast to other organoid cell lines, where the in vivo ratio of differentiated cells much more closely resembles the in vivo situation. Mature hepatocytes are the primary cell type responsible for hepatic metabolism of drugs. It is, therefore, critical to develop robust culture protocols to promote the differentiation of liver-derived ASC into more functionally active hepatocyte-like structures that would be more useful than cholangiocyte-dominated cultures for the study of drug metabolism and toxicity testing.

In this example, 24 different combinations of media compositions were investigated and the organoids from the resulting cell cultures were evaluated for characteristic morphologic and expression patterns pertaining to hepatic stem cells, as well as mature hepatocytes, and mature cholangiocytes. Furthermore, the functional ability of the organoid cell lines to produce albumin, as well as drug transporters and metabolic enzymes critical for mature hepatocyte function was investigated.

Overall, four major components were added to the differentiation media: BMP7 in 5/6 protocols, FGF19 in 5/6 protocols, and DAPT and Dex in all protocols. Additionally, specific media constituents were removed from the media to allow differentiation into functional hepatocytes. Among those, ROCKi was removed from 2/6 protocols, Nicotinamide from 3/6 protocols, and R-spondin-1 from 4/6 protocols. Partial differentiation into hepatocyte-like cells typically occurred on Day 3 to 6, with complete differentiation established for all culture conditions by Day 14.

Organoid morphology was assessed under bright field microscopy and shown to be markedly different between the experimental media and the control group. Specifically, instead of typical spheroids and budding organoids as seen in most epithelial ASC-derived mammalian organoid cultures, a more interconnected pattern with epithelial-to-mesenchymal transition around the extracellular basement membrane border was observed with the experimental media. Previous reports have noted the occurrence of stagnant organoid expansion, which was also the case in this example: On Days 7-14, apoptosis of organoids that was centered around the basement membrane was observed, with the outer cells appearing to have transitioned from an epithelial to a more mesenchymal phenotype, and the latter cells continuing to expand.

Measurement of albumin concentration in the culture supernatant was used as a screening assay to determine the functionality of mature hepatocytes. Six media with an average albumin production of >100 ng/mL were subsequently selected for additional experiments. Components for these high albumin-yielding media included: DAPT in all media, Dex in 5/6 media, BMP7 in 4/6 media, and FGF19 in 3/6 media. For comparison, the albumin yield of the supernatants in control-media cultured organoids was 15 to 111 times lower than the cutoff value for the second experiment. The hepatocyte differentiation media composition and the individual components and their combinations were further investigated. Each media component (DAPT, BMP7, FGF19, Dex) seemed to have limited influence on albumin production when used individually, and this observation was made both with media containing R-spondin and with media where R-spondin had been withdrawn. Higher production of albumin by functionally active hepatocytes was observed when using 2-, 3-, and 4-component media.

N-[N-(3,5-Difluorophenacetyl)-L-alanyl]-S-phenylglycine t-butyl ester (DAPT) acts as a γ-secretase inhibitor and an indirect Notch inhibitor. The main effect of DAPT likely lies in the inactivation of Notch signaling; which leads to an anti-proliferative effect on hepatocytes. DAPT seemed to be an important factor driving hepatocyte differentiation when added to BMP7 and Dex but lacked this effect when combined with FGF19. A combination of DAPT and Dex resulted in the highest albumin-yielding medium, with the addition of FGF19 not improving the albumin yield any further. FGF19 on the other hand prevented apoptosis in the experimental cultures.

Addition of Growth Factor 19 (FGF19) in the media for hepatocyte culture has previously been described to decrease albumin yield when added to a combination of BMP7 and DAPT. FGF19 is a ligand of FGF Receptor 4 (FGFR4), a mediator of hepatocyte proliferation, and suppressor of bile acid synthesis. Knockout of the fibroblast growth factor receptor 4 (FfzGFR4) gene was shown to provide anti-tumor benefits in a murine model of hepatocellular carcinoma. The protein activates the PI3K-AKT pathway, Ras/Raf/MAPK leading to anti-apoptotic properties and increased cellular proliferation and migration.

The combination of all four components yielded significant albumin concentrations of 757±624 ng/mL in the supernatant of our organoid cell lines when R-spondin was simultaneously removed from the culture. However, the albumin concentration achieved in the supernatant using this protocol was still lower than when BMP7 with DAPT or DAPT with Dex were combined altogether. These data suggest a neutral to negative effect of FGF19 addition on enhancing hepatocyte differentiation in canine hepatic organoids. The results also indicate that DAPT may play a crucial role in hepatocyte differentiation of canine organoid cultures. DAPT did not enhance differentiation when used by itself, however, differentiation was achieved when DAPT was combined with BMP7 and Dex. Of note, a low cell count of the differentiation and control samples was detected after the experiment, which was likely caused by lack of organoid upkeep due to experimental conditions. Surprisingly, however, this was not observed in the M01-treated organoid culture that achieved 5-fold expansion during their 14-day differentiation period.

Hepatocyte-derived plasma proteins were the main products identified in the M01 culture media (e.g., FETUB, CFL2, and AFM). These proteins are derived from functionally mature hepatocytes. This indicates the presence of functionally mature hepatocytes in culture.

Proteins that were found to be enriched in the supernatant of the control group cultures as compared to M01-treated organoids included Fibulin-1, with an abundance ratio 20 times higher than in control cultures. Cholangiocytes preferentially express this glycoprotein which functions to stabilize extracellular matrix, while Fibulin-1 expression has not previously been reported in canine differentiated hepatocytes. SPP1 is a marker of ductal cells and is commonly expressed in cholangiocytes. LAMC1, an extracellular matrix component typically expressed by cholangiocytes and other molecules of the laminin family, has been previously shown to drive cholangiocyte differentiation. VNT, CFI, LAMB1, CFB, and RBP are expressed in both hepatocytes and cholangiocytes.

CYP2B11 is a homolog of human CYP2B6, and its expression is several times higher in canine than in human liver and serves as a marker of hepatocyte function. Gene expression of CYP2B11 was statistically significantly different in the organoid treated with combination media of all components (M01) and the media composition containing DAPT+Dex (M16) as compared to the control media-treated cell lines. CYP3A12 is another important marker of hepatocyte function in dogs and tended to be more highly expressed in all of the organoid cell lines incubated with experimental media as compared to the control organoids, with the combination of DAPT and Dex (M16) being superior to all other differentiation medias in stimulating CYP3A12 expression. LGR5 is a marker expressed by undifferentiated epithelial stem cells and, as expected, expression of this markers was significantly lower in all differentiation media as compared to the control group. Finally, SOX9 represents a marker of cholangiocytes/ductal cells, and most of the experimental media-treated organoids expressed significantly lower levels of this marker when compared to the control.

Taken together, stem cell and cholangiocyte-specific markers were expressed less in all experimental media as compared to the controls and indicate significant differentiation towards mature hepatocytes when using the experimental media. Furthermore, this finding also suggests a crucial role of the addition of Dex for hepatocyte differentiation in organoid cultures.

Morphologically, most cells in the experimental cultures were described as phenotypically resembling cholangiocytes, except for the cultures derived from incubation with M01 and M20 media (with 10% mature hepatocytes) and M16 (with 5% mature hepatocytes). However, while the morphology of the majority of cells was mainly characterized as typical for cholangiocytes, our canine hepatic organoid cultures expressed significant amounts of hepatocyte-specific markers. This is notable, as achieving proper cellular morphology during hepatocyte differentiation in culture may be the most challenging part of hepatocyte organoid protocols. As expected, functional hepatocyte differentiation in the experimental cultures was observed using transmission electron microscopy images including an abundance of mitochondria, the presence of secretory vacuoles and bile canaliculi that had formed between hepatocyte-like cells. Finally, the presence and function of PGP was observed to occur mainly in the control sample, and points to the fact that the control cell lines mainly contained functional cholangiocytes, as PGP in the canine liver is mainly located in cholangiocytes.

In summary, HGF and FGF10 were added to the experimental culture media compositions, and ROCKi, CHIR99021, and Nicotinamide were removed from the media to encourage hepatocyte differentiation. DAPT and Dex have been shown to be major drivers of hepatocyte differentiation. The live cell count in cultures derived from incubation in media M16 and Mll was 2 times and 3 times higher than in control samples, respectively. On the other hand, M01 (containing BMP7, DAPT, FGF19, and Dex) produced 15 times more viable cells than the control group. The combination of all factors resulted in improved functionality of the organoids to secrete albumin, as well as a partial morphological differentiation into hepatocytes (10% mature hepatocytes). Cell cultures that had been treated with M01 media further demonstrated that the FGF19 component in media strongly supports the culture of ASC-derived hepatocytes by reducing the likelihood of apoptosis and promoting overall cell proliferation. FGF19 was not found to support hepatocyte differentiation in our experimental media compositions. Finally, these results highlight the necessity of removing R-spondin to allow proper hepatocyte differentiation in the canine organoid system.

### Example 4

As depicted in FIG. 5, an exemplar workflow for the transwell seeding protocol (“TSP”) is provided below. Said workflow includes a precoating of transwell inserts, canine organoid dissociation, canine organoid seeding, TEER Value Measurement, and monolayer upkeep.

### 1. Precoating of Transwell Inserts

1.1 Prepare an ice bucket and begin thawing Matrigel on ice. Place a 24-well plate containing the needed number of inserts in the incubator to pre-warm. Collect 1% collagen I, rat tail (3 mg/mL) and place on ice while protecting from light.
1.2 Pre-warm Complete media with growth factors enhanced with Rock inhibitor and GSKir3 (CMGF+ R/G) in the heat bath (37° C.). Furthermore, calculate the total number of inserts and blanks required for the experiment. Each insert will need 100 μL of gel Master Mix.
1.3 In a 15 mL tube, mix CMGF+ R/G with Matrigel (1%) and collagen I (1%) and gently pipet mix.
1.4 Coat each polyester insert with 100 μL of the gel Master Mix and place in the incubator (37° C.; 5% CO₂ atmosphere) for 1 hour.
1.5 After the incubation, carefully aspirate the gel Master Mix off each insert, being careful not to disturb the insert filter. Place a pre-coated plate in the incubator to keep warm.

### 2. Canine Organoid Dissociation

Notably, use of canine organoids that have been cultured for at least four days. Before beginning dissociation, refer to our other protocol to determine when a sample is healthy, dense, and sufficient for experimentation (Gabriel et al., 2022). It is recommended to dissociate one extra well of organoids for every well plating procedure. Furthermore, we recommend increasing the desired number of inserts by ˜20% to account for uneven organoid growth or damage caused by improper manipulation. If planning to use FITC-dextran, prepare extra wells.
2.1 Prepare an ice bucket and cold Advanced DMEM/F12 in the biosafety cabinet.
2.2. Place Matrigel on ice to begin thawing. Submersion in ice helps protect against rapid thawing and helps avoid solidification. A box of pipette tips can be placed in the freezer to assist in the plating of Matrigel.
2.3. Prechill a refrigerated centrifuge to 4° C.
2.4. Move CMGF+ R/G from the freezer/refrigerator to a 37° C. water bath. Avoid direct light exposure when possible.
2.5. Remove all media from wells for an appropriate number of wells (1 well of 24-well plate per 2-4 inserts) while taking care to not disturb the Matrigel.
2.6. Add 0.5 mL of pre-chilled Cell Recovery Solution per well to dissolve Matrigel domes.
2.7. Incubate the plate in the refrigerator (4° C.) for 30 min.
2.8. Pipette the suspension, collect all organoids and dissolved Matrigel, and transfer to a 15 mL tube.
2.9. Spin the tube (700×g for 5 min at 4° C.) and remove supernatant down to the 0.5 mL mark, making sure not to disturb the pellet.
2.10. Add 1 mL of TrypLE Express and incubate in the 37° C. water bath for 8 min. Flick the tube several times in the middle of incubation to mix the cells.
2.11. Move the tube containing the sample back to a biosafety cabinet and slowly add 7 mL of prechilled Advanced DMEM/F12 to inactivate TrypLE Express and stop the dissociation of the cells.
2.12. Gently pipet the mixture and filter the suspension through a 40 μm cell strainer.
2.13. Centrifuge the tube (700×g for 5 min at 4° C.) and remove the supernatant. Make certain not to disturb the pellet.
2.14. Resuspend the cell pellet in ˜300 μL culture media (CMGF+ R/G).
2.15. Count a subsample of the suspension (˜10 μL) with a hemocytometer or appropriate machine and determine the total cells in the suspension.

### 3. Canine Organoid Seeding

3.1 Dilute or concentrate the cell suspension to obtain a cell concentration of 75,000 cells per mL.
3.2 Seed 100 μL of the suspension into each insert.
3.3 Gently swirl the plate in a circular motion for ˜30 s to disperse the seeded cells across the insert.
3.4 Add 700 μL of CMGF+ R/G to the basolateral chamber and place the plate in the incubator (37° C.; 5% CO₂ atmosphere) for 24 hours.
3.5 After 24 hours, gently remove the cell suspension from the apical chamber and replace it with 200 μL of CMGF+ R/G. Return the plate to the incubator.

### 4. TEER Value Measurement

In embodiments, TEER value measurements are performed using an epithelial Volt/Ohm meter. TEER values provide information on the integrity of the canine organoid monolayer. TEER values are measured using electrodes (probes) and a volt/ohm meter. Probes must be chemically sterilized with 70% alcohol prior to inserting into the wells. The blank and organoid cell inserts are measured, and TEER values are calculated. Media is subsequently refreshed in both apical and basolateral chambers, and the canine organoid culture on the insert is visualized using light microscopy. Tears in either organoid culture or microporous membrane are noted and handled according to the protocol.
4.1. TEER value measurements are taken three days a week (e.g., on Mondays, Wednesdays, and Fridays) during cell culture growth.
4.2. Epithelial Volt/Ohm meter and its electrodes are moved to the biosafety cabinet. Electrodes are chemically sterilized in 70% alcohol before use. Wait at least one minute until the electrodes dry.
4.3. Before taking the first measurement, insert the wire electrode into the port and turn the power on. The meter should display 1000 SI If it is not the case, the device must be adjusted.
4.4. Insert electrodes in the apical and basolateral chamber of the cell-free insert (blank), so the apical chamber contains the shorter electrode, and the basolateral chamber contains the longer electrode. Make sure not to touch the membrane, but at the same time, electrodes must be submerged in the media.
4.5. Wait a few seconds until the value stabilizes and note the value in a lab book. Measure the remaining canine organoid monolayers, making sure to sterilize the electrodes with 70% alcohol when measuring different samples. Take care not to touch the organoid monolayer with the electrode.
4.6. After measurements are taken, sterilize the electrodes with 70% alcohol for the last time. Be sure to protect them from damage caused by inappropriate manipulation and storage according to manufacturer's instructions.
4.7. Calculate the TEER values for every well using the following formula where Rsampie and Rblank are the ohm (n) values from the monolayer and blank wells, respectively, and the area (cm²) is that of the insert.

TEER (Ω×cm²)=(R_{Sample}−R_{Blank})×Area [cm²]

### 5. Monolayer Upkeep

5.1. Using sterile disposable 9″ Pasteur pipets and a vacuum aspirator, gently aspirate the media from apical and basolateral chambers. Tilt the plate to see the media surface clearly. Avoid aspiration too close to the microporous membrane in the apical chamber to prevent damage to the cell monolayer. Notably, one should use a new Pasteur pipet when moving between samples.
5.2. Slowly add CMGF+RIG using P1000 pipettes aiming for a wall of the apical or basolateral chamber. Media change in the apical chamber should be performed very carefully to not damage the monolayer.
5.3. Wells are checked every other day under a light microscope, evaluating the health of the culture, and monitoring for tears in the organoid monolayer, or the microporous membrane. Using phase-contrast microscopy may highlight details of the culture.

Notably, in the case of canine organoid monolayer tears, the monolayer is given time to recover and regrow. In the case of microporous membrane tears, the well must be excluded from the experiment.

### Example 5

An exemplar workflow for a permeability experimental protocol summarized below. Said protocol is carried out subsequent to the five steps described in Example 3. Accordingly, the protocol steps are numbered 6 through 10 below, including the steps of evaluating organoid monolayer readiness, preparing for the experiment, typical experimental layout, 3D cell monolayer quality control, and fixing cell monolayers for downstream analysis. As described herein, said five permeability experimental protocol is used to measure a drug's in vitro permeability in hepatic organoids.

### 6. Evaluating Organoid Monolayer Readiness

In embodiments, these steps occur 8-14 days after seeding.
6.1. Check the monolayer at least every other day under the light microscope (phase-contrast may assist in the visualization of the monolayer's integrity). Continue to the next step when the cell monolayer is fully formed without gaps or apparent signs of tears.
6.2. Change organoid media from CMGF+ R/G to CMGF+ (excluding Rock inhibitor and GSKi(3 from the media composition). We recommend swapping the media at least four days prior to the experiment. The above two steps allow for proper differentiation of the organoid monolayer.
6.3. Continue measuring TEER values approximately every other day. When TEER values start to plateau at 2000 Ω×cm², measure TEER values every day. This steady state can be maintained for approximately 2-3 days, which is the optimal window of time to perform permeability testing.
6.4. The drug permeability assay must be scheduled immediately to avoid a rapid decrease in TEER values or overgrowth of the organoid monolayer to multiple cell layers.

### 7. Preparing for the Experiment

7.1. On the day of the experiment, measure TEER values and confirm that the values reached steady state and are not declining rapidly.
7.2. Choose the best monolayers (via light microscopy and TEER values) from the excess of 20% inserts to perform the experiment.
7.3. Observe monolayers under a light microscope and exclude incomplete, torn or overgrown organoid monolayers.
7.4. Prepare the transport buffer and adjust its pH to desired values.

In embodiments, the composition of the experimental buffer differs based on the experimental setup. A frequently used buffer is composed of Hank's Balanced Salt Solution (HBSS), glucose (12.5 mM), and 4-(2-hydroxyethyl)-1-piperazineethanesulfonic acid (HEPES, 25 mM). This composition ensures the viability of organoid culture during an experiment.
7.5. Carefully aspirate media from apical and basolateral chambers of selected wells.
7.6. Add 200 μL of transport buffer to the apical chamber and 800 μL to the basolateral chamber.
7.7. Place the plate in the incubator (37° C.; 5% CO₂ atmosphere) for 30 min to equilibrate.
7.8. The canine organoid monolayers are now ready for the drug permeability experiment.

### 8. Typical Experimental Layout

In embodiments, the experimental design and layout may change depending on the research question being asked. In one embodiment, multiple concentrations of the drug of interest are provided in 3-4 wells per group. The concentrations may be based on the expected intestinal and/or hepatic concentration of the drug. Furthermore, using previous research may help to determine appropriate time points for study design. A typical experiment collects samples in the receiver chambers over 2 hours (e.g., 15, 30, 60, 90, 120 minutes) Appropriate documentation of study design should be made to increase replicability and assist in troubleshooting.
8.1 Prepare the drug or solute by dissolving it in the transport buffer to the desired final concentration. Prepare more drug solution than needed. Drugs with low aqueous solubility may be first dissolved in an organic solvent (e.g., ethanol, DMSO) prior to adding to the buffer. The final concentration of the solvent should be less than 1% so as not to damage the cell monolayer.
8.2 Remove buffer from the donor chamber of each well.
8.3 Add the drug solution to all the donor chambers. The remaining solution serves as the time zero donor solution for measurement of initial drug concentration.
8.4 At the required time points, remove 50 μL from the receiving chamber and place it in a labeled tube. At the last timepoint, remove a sample from the donor chamber. At the end of the experiment, transfer the donor and receiver aliquots to a −20° C. freezer.

### 9. 3D Cell Monolayer Quality Control

FITC-dextran solution may be used to confirm monolayer integrity during the experiment.
9.1 At time 0 min, aspirate contents of the apical chamber and replace with 250 μL of FITC-dextran solution (XX) in triplicate for each experimental group. Do not expose FITC-dextran to light.
9.2 After 20 minutes, remove the buffer from the basolateral chamber.
9.3 Measure the fluorescence intensity of the basolateral sample using a fluorescence plate reader (excitation set at 485 nm and emission value at 528 nm).
9.4 After the experiment concludes, carefully aspirate excess buffer from the apical and basolateral chambers.
9.5 Add 200 μL of CMGF+ in the apical chamber and 700 μL to the basolateral chamber.
9.6 Measure TEER values in the individual wells.
9.7 Place the plate in the incubator (37° C.; 5% CO₂ atmosphere) for 24 hr.
9.8 If applicable, after 24 hours, measure TEER values to assess possible damage to the monolayer during the quality control portion of the experiment, use light microscopy to visualize the integrity of the canine organoid monolayer.

### 10. Fixing Cell Monolayers for Downstream Analysis

10.1 Prepare Formalin-Acetic Acid-Alcohol solution (See FAA composition in Table 2).
10.2 Fill the apical and basal chambers with FAA.
10.3 After 24 hours, aspirate the FAA and replace it with 70% Ethanol.
10.4 Wrap the plate with parafilm to prevent evaporation and proceed to block preparation.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof,

### Example 6

P-glycoprotein (P-gp) is encoded by the multi-drug resistance protein (ABCB1, MDR1) gene and is expressed in multiple tissues. Previous studies have shown that the MDR1 transporter is present on the apical side of the small intestine, large intestine, and also in the kidney, liver, and multiple other epithelial tissues. The expression of ABCB1 is also found in the blood brain barrier and is important in regulating the uptake of drugs into the central nervous system. P-gp also plays a crucial role in the liver, where it facilitates efflux of xenobiotics from cholangiocytes lining bile-facing canaliculi. A spontaneously occurring 4 base pair (bp) deletion mutation in the fourth exon (ABCB1-1Δ) in some dog breeds has been reported to result in a nonfunctioning P-gp protein in homozygous individuals, resulting from the formation of a premature stop codon. Homozygotic dogs for this deletion have increased sensitivity to loperamide, doxorubicin, vincristine, ivermectin and other xenobiotics. Canines that are heterozygous for the ABCB1-1Δ mutation can also have an increased sensitivity to vincristine and ivermectin. Furthermore, this mutation is not limited to a single dog breed. Approximately 75% of collies have at least one copy of this mutation, and the mutation is also present in longhaired whippets, Australian shepherds, McNab dogs, silken windhounds, old English sheepdogs, English shepherds, and Shetland sheepdogs. Additionally, any dog breed without genetic mutations in the ABCB1 gene can have acquired P-gp dysfunction.

Furthermore, genetic mutations affecting P-gp function are not limited to canines. In humans, it has been shown that compound heterozygotes of two nonsense mutations in ABCB1 can significantly impact the individual's ability to efflux parasiticides such as ivermectin. Therefore, in human medicine, genotyping of individuals prior to treatment can be performed to identify those carrying ABCB1 mutations.

The FDA recommends screening of drugs targeted for the human market for P-gp substrate specificity, both in vitro and in vivo in rodents and dogs. However, as explained above, certain dog breeds, including the most commonly used dog breed for research (Beagles) have an increased risk of carrying mutations in the ABCB1 gene. This can lead to false predictions for P-gp related drug transport when using in vivo dogs that carry the mutations. Furthermore, drugs targeted for the veterinary market should ideally be screened in vitro before being tested in vivo in healthy Beagles, to accurately predict P-gp substrate specificity as well as functional assessment of the transport properties of the drug.

Previous studies have generated a gene-edited 2D-Madin-Darby canine kidney (MDCK) cell line to investigate drug efflux enabled by the MDR1 transporter. However, this 2D cell line simply carries a mutation in the fourth exon of the ABCB1 gene, leading to a frameshift mutation, which results in a truncated protein. Therefore, this mutant 2D cell line does not contain the exact ABCB1-1Δ mutation that is naturally occurring in canines and does not lay a path to rescue the ABCB1-1Δ phenotype. Thus, there is a need for an accurate 3D organoid model capable of mimicking the mutation. In particular, there is a need for a method of mimicking this mutation without the use of a knock-in reporter or non-wild type DNA.

### Materials and Methods

Eleven dogs that were not client owned were screened for the 4 base pair deletion of ATAG in the fourth exon of the P-gp gene, known as ABCB1-1Δ. A Qiagen Blood and Tissue Kit was used on either whole blood samples or healthy liver organoid cultures to extract DNA. RNase A (Thermo Fisher Ref: EN0531) was added to degrade excess RNA. A Nanodrop was used to quantify the DNA, and a 1% agarose gel was run to ensure high molecular weight DNA. For PCR, Taq DNA Polymerase (Thermo Fisher Ref: 10342020) and dNTP mix (Thermo Fisher Ref: 18427013) were used. A Bio-Rad thermocycler was used with the following PCR conditions for the “PGP7”: Lid temp 105° C., 94° C. forever, 94° C. for 3 min, 94° C. for 30 s, 55° C. for 30 s [primer P4,P5,P6 used 54° C. for extension], 72° C. for 30 s, Go to step 3 for 30 times, 72° C. for 7 min, then 4° C. forever. PCR products were cleaned via the addition of 2μL of ExoSAPIT (Applied Biosystems) and incubated at 37° C. for 15 minutes and then 80° C. for 15 minutes to degrade excess dNTPs. Primers for Sanger sequencing were designed with Primer3 and can be seen in Table 4. Three primers were tested on one dog to identify the most reliable primer for screening. Samples were sequenced in the forward and reverse reactions as a long stretch of thymidines led to polymerase slippage; therefore, it was crucial to sequence in both directions. Reads were analyzed in Geneious Prime (v. 2020.2.4) and trimmed before or after the stretch of thymine, then the forward and reverse strands were aligned to create a consensus sequence. After eleven dogs were confirmed to not contain the ABCB1-1Δ mutation, the dog with the highest quality Sanger reads was used to design the HDR repair template.

### Plasmid Preparation

A plasmid containing a green fluorescent protein (GFP), specifically pEGFP-N1, was used to test transfection. Plasmid cultures were grown with 0.6 g of LB Broth Base (Invitrogen Ref: 12780052) in 30 mL of distilled water. The broth was mixed (appr. 3-5 minutes) then autoclaved for 20 min at 121° C. and allowed to cool to room temperature. One vial of One ShotTM TOP10 Chemically Competent E. coli (Thermo Fisher Ref: C404010) cells was thawed on ice, and 10 μL of the pEGFP-N1 plasmid was added. The vial was incubated for 30 min on ice, then heat shocked for 30 s at 42° C. It was placed on ice, and 250 μL of prewarmed SOC medium was added. The vial was shaken at 37° C. for 1 hour at 225 rpm in a Thermo Scientific Forma 420 Orbital Shaker. After shaking, 30 μL of Kanamycin Sulfate (Gibco Ref: 11815024) was added to the LB Broth, and the cell/plasmid mixture was added. Incubation took place overnight at 37° C., shaking at 250 rpm in a 50 mL vial. The next day the mixture was purified in a Qiagen EndoFree Plasmid Mega Kit to obtain transfection grade plasmid. The plasmid was quantified on a Nanodrop to confirm plasmid production, aliquoted, and stored at −20° C.

### Organoid Isolation and Culture

Necroptic whole tissue biopsies of the liver were harvested from a 29-day old female canine. After initial cultivation, healthy organoids were frozen in liquid nitrogen and then thawed for the experiment. Following thawing, the first passage of the organoids was incubated at 37° C. for 8 minutes to ensure recovery of the cells. Organoids were then cultured and expanded until there were 22 confluent wells in a 24 well plate.

Guide RNA (gRNA) Design

The gRNAs were designed using the Genescript (CRISPR sgRNA Design Tool) and CRISPOR (http://crispor.tefor.net) (Concordet & Haeussler, 2018) online resources. The crispr RNAs (crRNAs) and a universal trans-activating crispr RNA (tracrRNA) were ordered from IDT (Coralville, IA, USA) and their sequences are in Table 4. Simultaneous cutting of both guides without a repair template were confirmed on an agarose gel.

### Homology Directed Repair Template

The HDR template was a Megamer single-stranded DNA (ssDNA) fragment ordered from IDT. It was designed with 35 bp homology arms with a total length of the ssDNA being 337 bp. The HDR template used the exact sequence as the canine organoids were derived from, with the only difference being the 4 bp deletion [ATAG] near the middle of the strand. Both the positive and negative strand were reconstituted separately to determine which incorporation was most efficient.

TABLE 4
Sequences of primer, gRNA, and HDR oligos.
Homology arms are bolded.

| PCR Primers | 5′ Sequence 3′ | |
|---|---|---|
| MDR1_P4_F1 (SEQ ID NO: 1) | TGTCCCATTCCT | |
| | CTCATCAAAAC | |
| MDR1_P4_R1 (SEQ ID NO: 2) | TTTACCTCTTCC | |
| | TGAAACTTCCT | |
| MDR1_P5_F1 (SEQ ID NO: 3) | CCCATTCCTCTC | |
| | ATCAAAACTCA | |
| MDR1_P5_R1 (SEQ ID NO: 4) | TCCTTTACCTCT | |
| | TCCTGAAACTT | |
| MDR1_P6_F1 (SEQ ID NO: 5) | TCCCAGAATGTC | |
| | CCATTCCT | |
| MDR1_P6_R1 (SEQ ID NO: 6) | CAAGGTCTAGAT | |
| | AAGGTGGTTCC | |
| MDR1_P7_F1 (SEQ ID NO: 7) | GCAAACTATCC | |
| | ATGAAACTGTGC | |
| MDR1_P7_R1 (SEQ ID NO: 8) | TTTGCCAAGAC | |
| | CTCCAGTGT | |
| NGS Primers | 5′ Sequence 3′ | |
| GW_P7_F1 (SEQ ID NO: 9) | ACACTCTTTCCCTACACGACG | |
| | CTCTTCCGATCTGCAAACTAT | |
| | CCATGAAACTGTGC | |
| GW_P7_R1 (SEQ ID NO: 10) | GACTGGAGTTCAGACGTGTGC | |
| | TCTTCCGATCTTTTGCCAAGA | |
| | CCTCCAGTGT | |
| gRNAs | 5′ Sequence 3′ | PAM |
| PGP_F_1 (SEQ ID NO: 11) | GACTAATACT | TGG |
| | ATTACCATCC | |
| PGP_F_2 (SEQ ID NO: 12) | GCTTGATAGG | TGG |
| | TTGTATATGT | |
| PGP_R_1 (SEQ ID NO: 13) | GTCTGAGCTA | TGG |
| | CAGTTAATAT | |
| PGP_R_2 (SEQ ID NO: 14) | ATTTGTGGCA | AGG |
| | CTGATCTTAG | |

| HDR Template | 5′ Sequence 3′ |
|---|---|
| Positive Strand | GTTTCGCTATTCAAA |
| (SEQ ID NO: 15) | TTGGCTTGATAGGTT |
| | GTATATGTTGGTGGG |
| | GACAATGGCTGCCAT |
| | CATCCATGGAGCTGC |
| | ACTCCCTCTCATGAT |
| | GCTGGTTTTTGGAAA |
| | CATGACAGCTTTGCA |
| | AATGCAGGAATTTCA |
| | AGAAACAAAACTTTT |
| | CCAGTTATAATTAAT |
| | GAAAGTAAGTATTAT |
| | TTGTGGCACTGATCT |
| | TAGAGGTTTGAAGAA |
| | AAATCTGTTACTTAG |
| | AAGGATGTTTAGCCC |
| | TCATTAAATACATTA |
| | GTGTGTTTATTTCTG |
| | GGGGAAAAGGGAGGA |
| | GTCTGAGCTACAGTT |
| | AATATTGGTAATAAG |
| | TTTGAGAAGATAATA |
| | ATCATAA |
| Negative Strand | CAAAGCGATAAGTTT |
| (SEQ ID NO: 16) | AACCGAACTATCCAA |
| | CATATACAACCACCC |
| | CTGTTACCGACGGTA |
| | GTAGGTACCTCGACG |
| | TGAGGGAGAGTACTA |
| | CGACCAAAAACCTTT |
| | GTACTGTCGAAACGT |
| | TTACGTCCTTAAAGT |
| | TCTTTGTTTTGAAAA |
| | GGTCAATATTAATTA |
| | CTTTCATTCATAATA |
| | AACACCGTGACTAGA |
| | ATCTCCAAACTTCTT |
| | TTTAGACAATGAATC |
| | TTCCTACAAATCGGG |
| | AGTAATTTATGTAAT |
| | CACACAAATAAAGAC |
| | CCCCTTTTCCCTCCT |
| | CAGACTCGATGTCAA |
| | TTATAACCATTATTC |
| | AAACTCTTCTATTAT |
| | TAGTATT |

### Electroporation

Approximately 500,000 cells were pooled for each electroporation cuvette. Media was removed from the wells, and organoids were resuspended in 500 μL Advanced DMEM/F12 (Gibco; Ref 12634-010). Organoids were centrifuged at 700 g at 4° C. for 5 min to form a pellet. Media was removed down to 500 μL, and the pellet was resuspended with 500 μL of TrypLETM Express (Gibco, Ref 12604-021) and pipette mixed approximately 15 times to assist in dissociation. Organoids were immediately placed in the hot water bath for 10 minutes at 37° C. and halfway through they were briefly (1 s) vortexed twice. After incubation, the sample was pipette mixed (5 times) and 10 μL of the sample was taken to determine cell concentration and check for successful dissociation. As described in Merenda et al. 2017, clusters of 10-15 cells increased survival post electroporation compared to a single cell suspension (Merenda et al., 2017). Immediately 6 mL of Advanced DMEM/F12 was added to stop the chemical dissociation. Again, organoids were centrifuged at 700 g at 4° C. for 5 min to form a pellet and the supernatant was removed.

During dissociation, the ribonucleoprotein (RNP) was prepared. 150 pmol of crRNA (IDT) and 150 pmol of tracrRNA (IDT) were mixed and heated for 5 min at 95° C. in a thermocycler. Next, the mixture was removed from the heat and allowed to cool to room temperature for ˜10 minutes. Then, 300 pmol of Cas9 Nuclease V3 (IDT) was added and incubated for 10 min at room temperature (RT).

TABLE 5
The cuvette Master Mixes used for transfection.
Ribonucleoprotein Master Mix

| Reagent | Volume (μL) | Total pmol |
|---|---|---|
| tracRNA | 1.5 | 150 |
| crRNA | 1.5 | 150 |
| Cas9 | 4.92 | 300 |
| Total | 7.92 | |

Transfection Master Mix

| Reagent | CRISPR cuvette (μL) | GFP cuvette (μL) |
|---|---|---|
| Organoids | 10 | 10 |
| BTX solution | 78.08 | 87.01 |
| RNP complex | 7.92 × 2 | — |
| HDR template | 4 | — |
| GFP Plasmid | — | 10.91 |
| Total | 107.92 | 107.92 |

The organoid pellet was resuspended in 90 μL of BTXpress electroporation solution (BTX; Ref 45-0801). Then, 10 μL of plasmid/DNA was added to a 1.5 mL tube and gently pipette mixed as seen in Table 5. The solution was added to a chilled cuvette and after the addition of the solution, the cuvette was checked for bubbles, the outside was wiped dry, and placed in a NEPA21 electroporator (Nepagene). The electroporation details can be seen in Table 6. A control GFP plasmid was electroporated separately to help determine the efficiency of electroporation. It is common to add a buffer or media solution to assist in cell recovery after electroporation. Immediately following electroporation, 400 μL of room temperature CMGF+ R/G organoid media as previously described was added to the cuvette, and the organoids were transferred to a 1.5 mL tube. The cell mixture was incubated for 30 min at RT then centrifuged at 700 g for 5 min to form a pellet. The supernatant was removed, and the pellet was resuspended in Matrigel® Matrix (Corning, Ref 356231, 356255). The suspension was then pipetted into 30 drops per well. The plate was incubated at 37° C. and 5% CO₂ for 15 min to harden the Matrigel® Matrix, then 500 μL of media (CMGF+ R/G) was added. Then 5μL of Alt-R HDR enhancer (IDT; Ref 1081072) was added to wells containing an RNP. Half of each sample was placed in a 37° C. and 5% CO₂ incubator and the other half was placed at 30° C. and 5% CO₂ incubator for 48 hours to test “cold shock” before returning to 37° C. and 5% CO₂.

TABLE 6
The electroporation settings used on the NEPA21 electroporator

| Parameter | Poring Pulse | Transfer Pulse |
|---|---|---|
| Voltage (V) | 175 | 20 |
| Pulse Length (ms) | 5.0 | 50.0 |
| Number of Pulses | 2 | 5 |
| Pulse Interval (ms) | 50.0 | 50.0 |
| Decay Rate (%) | 10 | 40 |
| Cuvette (mm) | 2 | 2 |
| Polarity | + | +/− |

### Mutation Screening

Three days after transfection, organoids were collected via the addition of 500 of Advanced DMEM/F12 and centrifuged (700 g at 4° C. for 5 min) to form a pellet. Media was removed down to 500 μL and 500 μL of TrypLETM Express was added. The pellet was resuspended and mechanically dissociated by pipette mixing 10-20 times. The mixture was then placed in the hot water bath at 37° C. for 10 minutes and flicked halfway through. After 10 minutes it was removed and again vigorously mechanically dissociated. To stop dissociation, 6 mL of Advanced DMEM/F12 was added and then it was centrifuged (700 g at 4° C. for 5 min). The supernatant was removed, and cells were seeded using Matrigel® Matrix into two 24 well plates with each well replicated in the other plate.

Organoids were allowed to recover and expand before being used for DNA extractions. After expansion, a subset of the cultures was collected, and DNA extractions were performed as described earlier. The genomic DNA was used in a PCR reaction with new primers that have additional Illumina adapter sequences (GW_P7_F1 and GW_P7_R1) as seen in Table 4. Genewiz Amplicon-EZ sequencing was used to screen cultures for the proper insertion of the HDR template. The reads were first analyzed with FastQC for quality control. The bioinformatician was blinded to the sample treatments. The reads were then mapped to the reference using Bowtie2 using the -sensitive parameter. Sam files were processed using SAMtools and coverage was calculated using BEDTools. Average normal reads mapped is the number of reads mapped over the area of interest (deletion) in the reference. Two reference sequences were used, (1) the original sequence and (2) the sequence modified to match the deletion.

### Results

DNA was extracted and PCR products of the MDR1 gene were sequenced for eleven dogs. The region of interest (exon 4 in the MDR1 gene) was largely conserved showing the ability to use similar gRNAs and HDR templates across individuals.

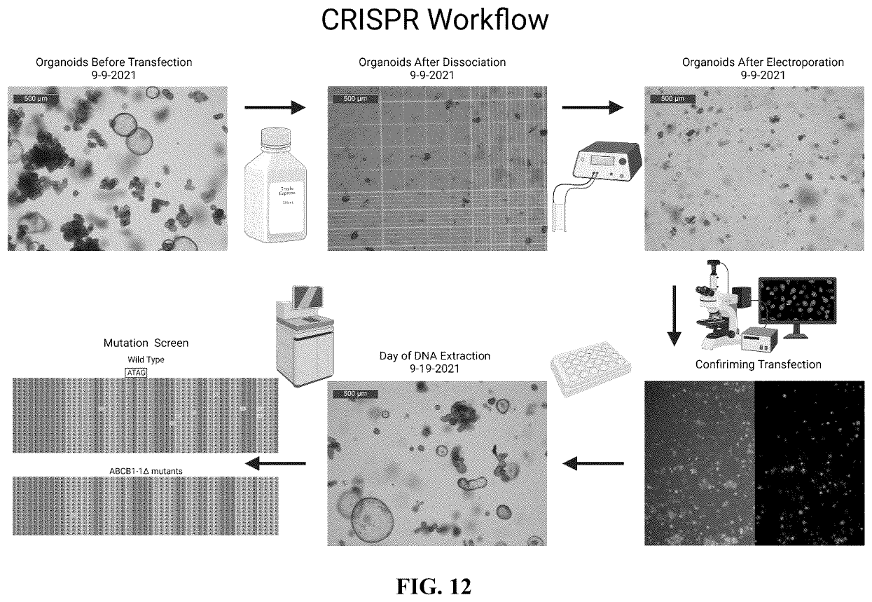

A proof-of-concept transfection was completed using two gRNAs. Post-transfection, two distinct bands were detectable on an agarose gel (FIG. 12), one WT band and one where both gRNAs had successfully cut and the region between them was spliced out. The truncated band formed by simultaneous cuts of two gRNAs to the WT band was estimated using Gel Analyzer and the ratio of the small band to the large band was ˜18.7%.

After confirming that gRNAs were functional, transfection was done with cuvettes containing the antisense strand and RNP, sense strand and RNP, or a plasmid. The pEGFP-N1 plasmid containing GFP was used to test transfection efficiency and can be seen in FIG. 12. Additionally, a subset of organoids was passaged at the same time but was excluded from electroporation and used as a control (Passage Control).

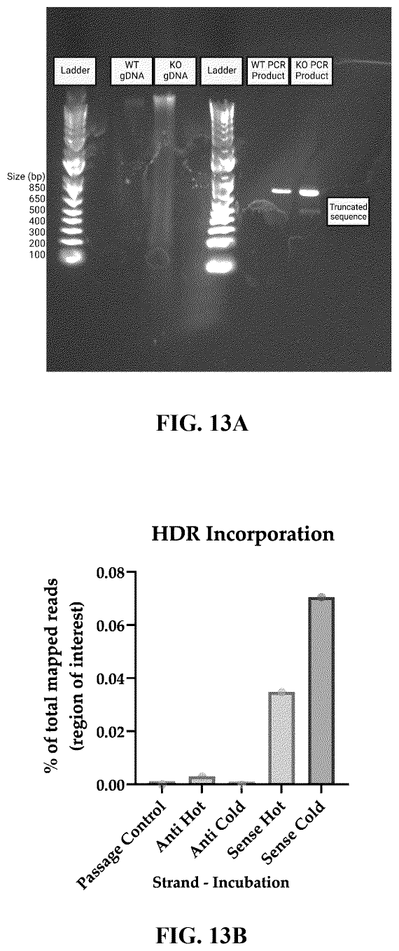

Successful editing was confirmed using agarose gel (FIG. 13A). The successful incorporation of the HDR template occurred with different frequencies across the samples. The “cold shock” technique was used on both the antisense and sense strands. Therefore, the samples are referred to as follows: antisense strand at 37° C. (Anti Hot), antisense strand at 30° C. (Anti Cold), sense strand at 37° C. (Sense Hot), sense strand at 30° C. (Sense Cold), and the wild-type control (Passage Control). The estimated efficiencies of the correct insertion can be seen in FIG. 13B and were: Passage Control 0%, Anti Hot ˜0.00303%, Anti Cold 0%, Sense Hot ˜0.0348%, and Sense Cold ˜0.0705%. The Anti Cold treatment resulted in drastically fewer reads compared to the other samples possibly due to sequencing errors, but no correct mutation was seen.

### Discussion

Finding an otherwise healthy dog with the (ABCB1-1Δ) mutation to harvest tissue from can prove difficult as genetic screenings are needed. Additionally, after a donor is found, if biopsies are being taken, further consent is needed for additional biopsies to be taken for use in organoid cultures. Screening of ten Beagle dogs in a research colony was completed, and this assessment confirmed that none of the dogs carried the mutation of interest, further demonstrating the difficulty of identifying donors. However, during this screening process the PCR primers were optimized for future studies. The most reliable PCR primer set for the initial screen of 10 dogs was MDR1_P5_F1 and MDR1_P5_R1 which amplified DNA in 9 out of 10 dogs. To increase the sequencing area for the HDR template, a new primer set made of MDR1_P7_F1 and MDR1_ P7_R1 was used for the organoid cultures in dog No. 11, which was the donor used for the gene editing experiments.

The typical CRISPR/Cas9 system relies on the need for a protospacer adjacent motif (PAM), specifically three base pairs (NGG), approximately three base pairs from the desired cut site. This limitation on the design of gRNAs is being addressed with modified Cas9s with different PAM requirements. Other CRISPR systems such as Cas12a have different PAM requirements but can cut with varying efficiencies. With the availability of aTAM site near the four bp ABCB1-1Δ mutation, it is possible to use one gRNA with a repair template. However, the four bp insertion/deletion would alter the sequence of the location from the originally targeted gRNA. To create the ABCB1-1Δ mutation, one would need one gRNA and one HDR template. Furthermore, to rescue the phenotype in organoids, a new gRNA (as the gRNA site is altered) and a new HDR template would be needed. Our experimental design would also rely on two gRNAs and two HDR templates; however, optimization of either gRNA can be done by choosing other gRNAs flanking the mutation. Reliance on a single gRNA is undesirable as the efficiency is not guaranteed and the lack of another NGG PAM flanking the ABCB1-1Δ mutation limits gRNA options. Furthermore, after confirming suitable gRNAs, the same two gRNAs will be unaffected by the HDR insertion and can be used to rescue the mutation. This protocol is applicable to other areas of research where a PAM is not near the site of interest and two gRNAs can be used to replace the genome with the desired HDR template.

Previous studies have completed knockouts of canine MDR1 in MDCK cell lines which led to nonfunctional mutants that were different than the naturally occurring ABCB1-1Δ previously described in canines. Specifically, previous methods included a single cut with a single gRNA. This led to a frameshift mutation which simply makes the rest of the protein nonfunctional. The present system, on the other hand, uses 2 gRNAs to precisely excise a strand of DNA and subsequently knock-in a new HDR strand containing the exact sequence with the mutation. Thus, the present system results in a complete mimic of the original wild-type mutation (ABCB1-1Δ). The importance of accurately mimicking the original wild-type mutation (ABCB1-1Δ) is crucial for potential downstream biomedical applications needing to study the correct phenotype. For this reason, the experimental design in this study used RNPs which cut the genomic DNA once and then dissociate, further reducing the risk of off-target mutations. Electroporation delivery, resuspension in BTXpress electroporation solution, and a NEPA21 electroporator were all chosen to increase transfection efficiency as previously demonstrated by Fujii et al 2015.

The effects of a colder incubation post transfection have previously been tested using Zinc-Finger Nucleases (ZFN), with increased activity detected when cells were incubated in colder conditions. Furthermore, studies have shown that “cold shock” can be used to increase HDR insertion when used in the CRISPR/Cas9 system. Here, the successful use of “cold shock” in CRISPR/Cas9 systems was also demonstrated within organoids, which greatly increased HDR incorporation in the samples using the Sense strand as seen in FIG. 13B. The cold shock method more than doubled the correct insertion of the template with no indels present.

Additional methods can be used to screen and increase the pool of desired clones or create a single clonal line when using genetic editing methods with relatively low efficiencies. To alleviate the problem of having a large pool with few mutants of interest, a fluorescent antibody can be used to target functional P-gp receptors allowing for them to be discarded to drastically increase the percentage of non-functional mutants via Fluorescence-activated cell sorting (FACS). Organoids can be expanded to large numbers, allowing for thousands of additional cells to be grown after electroporation and before FACS sorting to select hundreds to thousands of non-functional P-gp mutants. Preliminary tests of FACS sorting of gene-edited liver organoids has been completed as well as expanding organoids from single cells, showing that FACS is a viable route for clonal selection. In combination with reagents such as Label-IT® Nucleic Acid Labeling Reagents, gRNAs or ssDNA templates can be fluorescently tagged without affecting their sequence. This allows for the screening of potential mutants without inserting foreign DNA into the genome. Future studies can rescue this genotype as this two-gRNA design allows for the repair template to be switched while not modifying the gRNA target sites. Rescue of an individual or organoid line with the ABCB1-1Δ mutation can be performed using the ssDNA repair template with the addition of the ATAG sequence. This method of knocking out a sequence of DNA and replacing it with a desired repair template is applicable across systems.

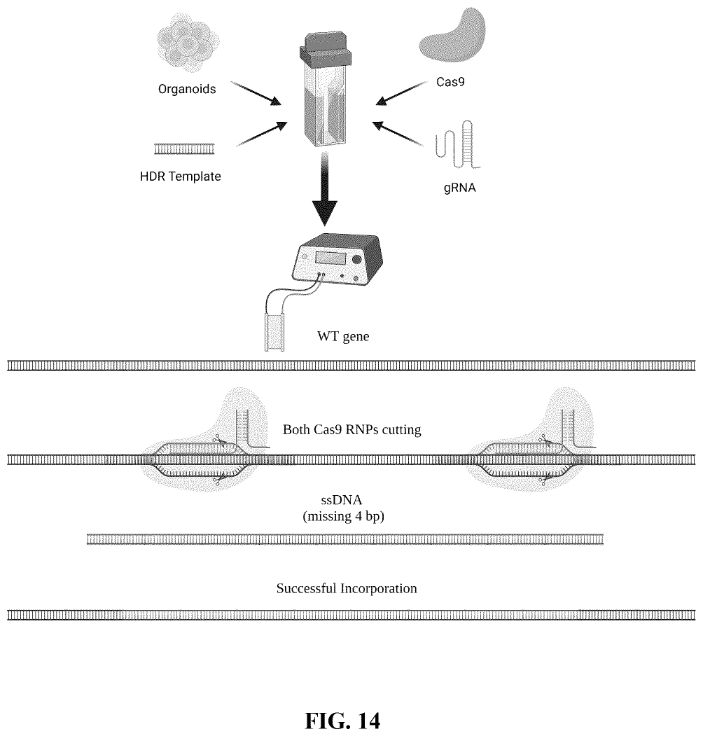

The genome editing technique described, as seen in FIG. 14, has been successfully applied in the hepatic organoid model. This technology has the potential to be applicable in the field of drug development, drug testing, and eventually, even regenerative medicine. The use of an expandable cell culture system such as organoids allows for a large pool of organoids to be edited at once. Through additional screenings, edited and screened organoids could be reintroduced back to the diseased donor for potential therapeutic uses. The method described herein, for the first time, allows editing of healthy canine organoids to a diseased phenotype and derive a direct comparison with healthy organoid cultures, to perform drug transport studies involving P-gp substrates. Previous studies lacked the creation of the correct ABCB1-1Δ mutant, therefore leading to an inaccurate model of the phenotypic effects in the presence of dysfunctional P-gp.

What is claimed is:

1. A canine hepatic organoid, comprising:
a population of differentiated canine hepatic-derived cells which are capable of organ-like functionality, wherein the hepatic organoid is genetically modified to alter the expression of P-glycoprotein (P-gp).

2. The hepatic organoid of claim 1, wherein the hepatic-derived cells are adult stem-cell derived cells.

3. The hepatic organoid of claim 1, wherein the hepatic-derived cells are induced pluripotent derived stem cells.

4. The hepatic organoid of claim 1, further comprising an extracellular matrix, wherein the hepatic organoid maintains the organ's three-dimensional structures.

5. The hepatic organoid of claim 4, wherein the hepatic-derived cells are epithelial cells and maintain the expression of tight junction proteins.

6. The hepatic organoid of claim 1, wherein the hepatic organoid is genetically modified to decrease expression of P-gp.

7. The hepatic organoid of claim 1, wherein the genetic modification comprises a knock-in mutation of an ABCB1-1Δ mutation.

8. The hepatic organoid of claim 1, wherein the epithelial-derived cells are diseased.

9. The hepatic organoid of claim 8, wherein the genetic modification is conferred using at least two gRNA molecules.

10. A culture media for differentiating hepatic stem cells into hepatic organoids, comprising:
a complete media; and
a growth factor, wherein the growth factor differentiates the hepatic stem cell into the hepatic organoid.

11. The culture media of claim 10, wherein the growth factor is epidermal growth factor, Noggin, DAPT, dexamethasone, fibroblast growth factor 10 (FGF10), fibroblast growth factor 19 (FGF19), bone morphogenic protein 7 (BMP7), hepatocyte growth factor (HGF), a transforming growth factor beta receptor I inhibitor, a mitogen activated protein kinase 14 inhibitor, and/or combinations thereof.

12. The culture media of claim 10, further comprising a rho kinase inhibitor and/or a glycogen synthase kinase 3 inhibitor.

13. The culture media of claim 12, wherein the rho kinase inhibitor is Y27632, Y39983, Wf-536, SLx-2119, Azabenzimidazole-aminofurazans, DE-104, olefins, isoquinolines, indazoles, pyridinealkene derivatives, H-1152P, ROKa inhibitors, XD-4000, HMN-1152, 4-(1-aminoalkyl)-N-(4-pyridyl)cyclohexane-carboxamides, Rhostatin, BA-210, BA-207, BA-215, BA-285, BA-1037, Ki-23095, VAS-012, fasudil and/or combinations thereof.

14. The culture media of claim 13, wherein the glycogen synthase kinase 3 inhibitor is an aminopyrimidine.

15. The culture media of claim 10, wherein one or more growth factors are removed from the culture media.

16. The culture media of claim 15, wherein the one or more removed growth factors is ROCKi, CHIR99021, Nicotinamide, R-Spondin, and/or combinations thereof.

17. A canine hepatic organoid culture system, comprising:
a three-dimensional canine epithelial organoid, comprising a population of differentiated canine epithelial-derived cells which are capable of organ-like functionality;
an extracellular matrix; and
the culture media of claim 10.

18. The method of claim 17, further comprising isolating a stem cell of interest from the hepatic sample; and enriching the sample for stem cells.

19. The method of claim 17, further comprising seeding the sample into an extracellular matrix.

20. The method of claim 19, wherein between about 20 to about 200 cells are seeded into the extracellular matrix.

21. The method of claim 20, wherein the extracellular matrix stabilizes the three-dimensional structure of the organoid.

22. The method of claim 17, further comprising initially contacting the sample with a protective media.

23. The method of claim 22 wherein the sample is contacted with the protective media for about 1 day to about 4 days.

24. The method of claim 22, wherein the protective media comprises a rho kinase inhibitor and/or a glycogen synthase kinase 3 inhibitor.

25. The method of claim 24, wherein the rho kinase inhibitor is Y27632, Y39983, Wf-536, SLx-2119, Azabenzimidazole-aminofurazans, DE-104, olefins, isoquinolines, indazoles, pyridinealkene derivatives, H-1152P, ROKa inhibitors, XD-4000, HMN-1152, 4-(1-aminoalkyl)-N-(4-pyridyl)cyclohexane-carboxamides, Rhostatin, BA-210, BA-207, BA-215, BA-285, BA-1037, Ki-23095, VAS-012, fasudil and/or combinations thereof.

26. The method of claim 24, wherein the glycogen synthase kinase 3 inhibitor is an aminopyrimidine.

27. The method of claim 17, wherein the growth factor is epidermal growth factor, Noggin, DAPT, dexamethasone, fibroblast growth factor 10 (FGF10), fibroblast growth factor 19 (FGF19), bone morphogenic protein 7 (BMP7), hepatocyte growth factor (HGF), a transforming growth factor beta receptor I inhibitor, a mitogen activated protein kinase 14 inhibitor, and/or combinations thereof.

28. The method of claim 17, further comprising genetic engineering the sample.

29. The method of claim 28, wherein the genetic engineering is performed prior to exposing the sample to the differentiation media.

30. The method of claim 28, wherein the genetic engineering is a DNA modification.

31. The method of claim 30, wherein the genetic engineering is performed by Cas variants, TALEN, meganucleases, or Zinc Fingers.

32. The method of claim 28, wherein the genetic engineering is a RNA modification.

33. The method of claim 28, wherein the genetic engineering is performed using at least two gRNA molecules.

34. A method of screening drug absorption in the canine liver, comprising:
obtaining a sample of canine liver;
genetically modifying the sample to alter the expression of P-glycoprotein (P-gp);
culturing the sample in differentiation media to form an organoid;
administering a drug;
allowing sufficient time for absorption into the organoid lumen; and
detecting the concentration of the drug in the lumen and/or intracellular space of the organoid.

35. The method of claim 34, further comprising administering a P-glycoprotein interacting compound to the organoid.

36. The method of claim 35, wherein the P-glycoprotein interacting compound is an inhibitor.

37. The method of claim 36, wherein the P-glycoprotein interacting compound is a substrate.

38. The method of claim 35, wherein the P-glycoprotein interacting compound is an inducer.

39. The method of claim 34, wherein two or more samples are obtained at different time points from the same subject.

40. The method of any one of claim 34, wherein detecting is measured by fluorescence, a binding assay, through high performance liquid chromatography, and/or staining.

41. The method of claim 34, wherein the genetic-modification decreases expression of P-glycoprotein.

42. The method of claim 34, wherein the genetic modification comprises a knock-in mutation of an ABCB1-1Δ mutation.

43. The method of claim 34, wherein the genetic-modification alters the binding kinetics of P-glycoprotein.

44. The method of claim 39, wherein the differentiation media comprises a growth factor.

45. The method of claim 44, wherein the growth factor is epidermal growth factor, Noggin, DAPT, dexamethasone, fibroblast growth factor 10 (FGF10), fibroblast growth factor 19 (FGF19), bone morphogenic protein 7 (BMP7), hepatocyte growth factor (HGF), a transforming growth factor beta receptor I inhibitor, a mitogen activated protein kinase 14 inhibitor, and/or combinations thereof.

46. The method of claim 34, wherein the genetic modification is conferred using at least two gRNA molecules.
